(19) United States
(12) Patent Application Publication
(10) Pub. No.: US 2007/0149499 A1
(43) Pub. Date: Jun. 28, 2007

(54) Process for preparing beta-lactamase inhibitors

(75) Inventors: Ronald S. Michalak, Congers, NY (US); Rocco Galante, Oakland, NJ (US); David M. Blum, Upper Saddle River, NJ (US); Julianne Blum, Upper Saddle River, NJ (US); Lisa Routel, Valley Cottage, NY (US); Haris Durutlic, New Windsor, NY (US); Charles Guinosso, Chestnut Ridge, NY (US); John Considine, Bridgewater, NJ (US); Kenneth A.M. Kremer, Lawrenceville, NJ (US)

Correspondence Address: WilmerHale/Wyeth, 60 STATE STREET, BOSTON, MA, 02109, US

(73) Assignee: Wyeth, Madison, NJ (US)

(21) Appl. No.: 11/508,037

(22) Filed: Aug. 22, 2006

Related U.S. Application Data
(60) Provisional application No. 60/710,957, filed on Aug. 24, 2005.

(51) Int. Cl. A61K 31/43; C07D 499/46

(52) U.S. Cl. 514/196; 548/154; 540/329

(57) ABSTRACT

The present invention relates to processes for preparing β-alkylidene penem derivatives that can be important as broad spectrum β-lactamase inhibitors and anti-bacterial agents.

Throughout this application, various publications are referenced. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure, as it appears in the U.S. Patent and Trademark Office patent file or records, but otherwise reserves any and all copyright rights whatsoever.

### FIELD OF THE INVENTION

The present invention relates to processes for preparing β-alkylidene penem derivatives that can be important as broad spectrum β-lactamase inhibitors and anti-bacterial agents.

### BACKGROUND OF THE INVENTION

β-Lactamases are enzymes produced by the bacteria, which hydrolyze β-lactam antibiotics and as such serve as the primary cause of bacterial resistance. Penicillins and cephalosporins are the most frequently and widely used β-lactam antibiotics in the clinic. However, the development of resistance to β-lactam antibiotics by different pathogens has had a damaging effect on maintaining the effective treatment of bacterial infections. (Coleman, K. Expert Opin. Invest. Drugs 1995, 4, 693; Sutherland, R. I 1995, 23(4), 191; Bush, K. Curr. Pharm. Design 1999, 5, 839-845). The most significant known mechanism related to the development of bacterial resistance to the β-lactam antibiotics is the production of Class-A, Class-B, and Class-C serine β-lactamases. These enzymes degrade the β-lactam antibiotics, resulting in the loss of antibacterial activity. Class-A enzymes preferentially hydrolyze penicillins whereas Class-C lactamases have a substrate profile favoring cephalosporin hydrolysis. (Bush, K., Jacoby, G. A., Medeiros, A. A., Antimicrob. Agents Chemother. 1995, 39, 1211). To date over 250 different β-lactamases have been reported (Payne, D. J., Du, W. and Bateson, J. H., Exp. Opin. Invest. Drugs 2000, 247) and there is a need for a new generation of broad spectrum β-lactamase inhibitors. Bacterial resistance to these antibiotics could be greatly reduced by administering the β-lactam antibiotics in combination with a compound that inhibits these enzymes.

The commercially available β-lactamase inhibitors such as clavulanic acid, sulbactam and tazobactam are all effective against Class-A producing pathogens. Clavulanic acid is used in combination with amoxicillin and ticarcillin; similarly sulbactam with ampicillin and tazobactam with piperacillin. The mechanism of inactivation of Class-A β-lactamases (such as PCI and TEM-1) has been elucidated. (Bush, K. Antimicrob. Agents Chemother. 1993, 37, 851; Yang, Y., Janota, K., Tabei, K., Huang, N., Seigal, M. M., Lin. Y. I., Rasmussen, B. A. and Shalaes, D. M. J. Biol. Chem. 2000, 35, 26674-26682). However, these compounds are ineffective against Class-C producing organisms.

Accordingly, the need exists for β-lactamase inhibitors, methods of synthesizing β-lactamase inhibitors, and methods of synthesizing intermediates useful in the preparation of β-lactamase inhibitors.

The present invention is directed to these and other important ends.

### SUMMARY OF THE INVENTION

In one embodiment, the invention provides methods of synthesizing a compound of the formula (II) 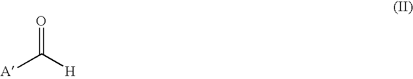
or a pharmaceutically acceptable salt thereof,

wherein A′ is 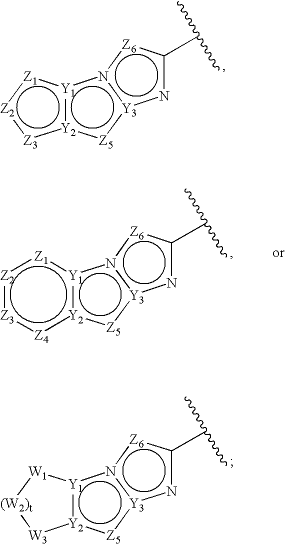
and wherein Z₁-Z₆, Y₁—Y₃, W₁—W₃, and t are as defined below.

In one embodiment, the methods of the invention comprise reacting a compound of the formula (III)
D-NH₂ (III)
or a pharmaceutically acceptable salt thereof,

wherein D is 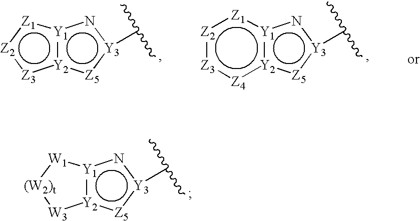
and wherein Z₁-Z₅, Y₁—Y₃, W₁—W₃, and t are as defined below, under conditions effective to bring about cyclization, thereby providing an aldehyde of the formula (II).

In another embodiment, the invention provides methods of synthesizing a compound of the formula (II) 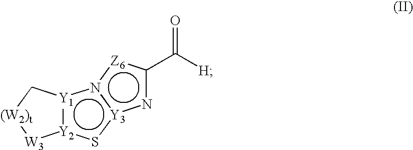
or a pharmaceutically acceptable salt thereof,

wherein Z₆, Y₁—Y₃, W₂—W₃, and t are as defined below.

In one embodiment, the methods of the invention comprise reacting a compound of the formula (VII) 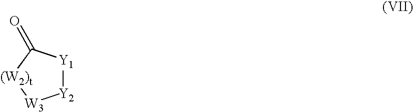
or a pharmaceutically acceptable salt thereof,

under conditions effective to bring about cyclization, thereby providing an amine of the formula (III) 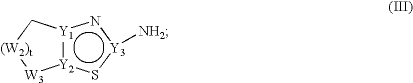
or a pharmaceutically acceptable salt thereof,

and reacting a compound of the formula (III) or a pharmaceutically acceptable salt thereof, under conditions effective to bring about cyclization, thereby providing an aldehyde of the formula (II) or a pharmaceutically acceptable salt thereof; wherein Y₁—Y₃, W₂—W₃, and t are as defined below.

In one embodiment, the invention provides methods of synthesizing a compound of the formula (VII) 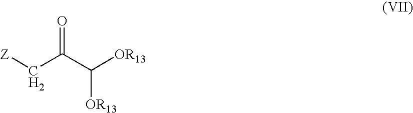
wherein Z is halogen, such as bromine, chlorine or iodine, and R₁₃ is an optionally substituted alkyl or aralkyl group, by reacting a compound of the formula (VIII) 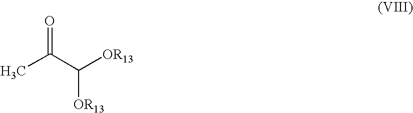
in the presence of a halogenating agent and from about 1% to about 100% (v/v) methanol in acetonitrile, thereby providing a compound of the formula (VII).

In one embodiment, the invention provides methods of synthesizing a compound of the formula (I) 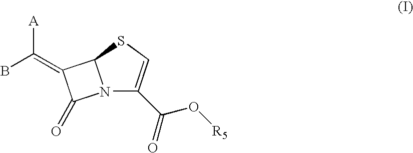
or a pharmaceutically acceptable salt thereof,

wherein A, B, and R₅ are as defined below.

### DESCRIPTION OF THE INVENTION

Definitions

The term “(C₁-C₆)-alkyl” as used herein refers to a linear or branched, saturated hydrocarbon having from 1 to 6 carbon atoms; in one embodiment, from 1 to 5 carbon atoms; in one embodiment, from 1 to 4 carbon atoms; in one embodiment from 1 to 3 carbon atoms; in one embodiment, from 1 to 2 carbon atoms; in one embodiment, 1 carbon atom. Representative (C₁-C₆)-alkyl groups include, but are not limited to, methyl, ethyl, propyl, isopropyl, butyl, sec-butyl, tert-butyl, pentyl, isopentyl, neopentyl, hexyl, isohexyl, and neohexyl. In one embodiment, the (C₁-C₆)-alkyl group is optionally substituted. Accordingly, a “(C₂-C₄)-alkyl” group as used herein refers to a linear or branched, saturated hydrocarbon having from 2 to 4 carbon atoms, and which may be optionally substituted as indicated for a (C₁-C₆)-alkyl group.

The term “(C₂-C₆)-alkenyl” as used herein refers to a linear or branched hydrocarbon having from 2 to 6 carbon atoms and having at least one carbon-carbon double bond; in one embodiment, from 2 to 5 carbon atoms; in one embodiment, from 2 to 4 carbon atoms, in one embodiment; from 2 to 3 carbon atoms; in one embodiment, 3 carbon atoms. In one embodiment, the (C₂-C₆)-alkenyl has one or two double bonds. The (C₂-C₆)-alkenyl moiety may exist in the E or Z conformation and the compounds of the present invention include both conformations. In one embodiment, the (C₂-C₆)-alkenyl group is optionally substituted. Similarly, a “(C₂-C₈)-alkenyl” group as used herein refers to a linear or branched, saturated hydrocarbon having from 2 to 8 carbon atoms, and which may be optionally substituted as indicated for a (C₂-C₆)-alkenyl group.

The term “(C₂-C₆)-alkynyl” as used herein refers to a linear or branched hydrocarbon having from 2 to 6 carbon atoms and having at least one carbon-carbon triple bond; in one embodiment, from 2 to 5 carbon atoms; in one embodiment, from 2 to 4 carbon atoms, in one embodiment; from 2 to 3 carbon atoms; in one embodiment, 3 carbon atoms. In one embodiment, the (C₂-C₆)-alkenyl group is optionally substituted.

The term “protecting group” as used herein refers to a moiety that temporarily blocks a reactive site in a compound, such as a carboxyl group, an alcohol, or an amine. Generally, this is done so that a chemical reaction can be carried out at another reactive site in a multifunctional compound or to otherwise stabilize the compound. In one embodiment, the protecting group is selectively removable by a chemical reaction. An exemplary carboxyl protecting group is an ester group. Ester protecting groups include, without limitation, benzyl, p-nitrobenzyl, p-methoxybenzyl, triphenylmethyl (trityl), and benzylhydrol. See, Greene and Wuts, Protecting Groups in Organic Synthesis, Second Edition, John Wiley & Sons (1991).

The term “aryl” as used herein refers to an aromatic hydrocarbon moiety, e.g., 6-14 carbon atoms, for example selected from phenyl, α-naphthyl, β-naphthyl, biphenyl, anthryl, tetrahydronaphthyl, fluorenyl, indanyl, biphenylenyl, and acenaphthenyl. In one embodiment, the aryl group is optionally substituted.

The term “conditions effective to” as used herein refers to synthetic reaction conditions which will be apparent to those skilled in the art of synthetic organic chemistry.

The term “cycloalkyl” as used herein refers to a three- to seven-membered saturated or partially unsaturated carbon ring, unless specified otherwise; in one embodiment, 7 carbon atoms; in one embodiment, 6 carbon atoms; in one embodiment 5 carbon atoms; in one embodiment, 4 carbon atoms; in one embodiment, 3 carbon atoms. Any suitable ring position of the cycloalkyl group may be covalently linked to the defined chemical structure. Exemplary cycloalkyl groups include cyclopropyl, cyclobutyl, cyclopentyl, cyclohexyl, and cycloheptyl. In one embodiment, the cycloalkyl group is optionally substituted.

The term “halogen” as used herein refers to fluorine, chlorine, bromine, and iodine.

The term “heteroaryl” as used herein refers to an aromatic heterocyclic ring system having one or two rings, e.g., having 5-14 ring members (in some embodiments, 5-10 ring members, in some embodiments, 5-8 ring members) and 1-3 heteroatoms selected from O, N, and S. Exemplary heteroaryls include, without limitation: (1) furan, furazanyl, thiophene, indole, azaindole, oxazole, thiazole, isoxazole, isothiazole, imidazole, N-methylimidazole, pyridine, pyrimidine, pyrazine, pyrrole, N-methylpyrrole, pyrazole, pyrimidinyl, pyridooxazolyl, pyridoimidazolyl, pyridothiazolyl, pyridazinyl, thienyl, thienothiazolyl, thienooxazolyl, thienoimidazolyl, N-methylpyrazole, 1,3,4-oxadiazole, 1,2,4-triazole, 1-methyl-1,2,4-triazole, 1H-tetrazole, 1-methyltetrazole, benzoxazole, benzothiazole, benzofuran, benzisoxazole, benzimidazole, N-methylbenzimidazole, azabenzimidazole, indazole, quinazoline, quinoline, and isoquinoline; (2) a bicyclic aromatic heterocycle where a phenyl, pyridine, pyrimidine, or pyridizine is: (a) fused to a 6-membered aromatic heterocyclic ring having one nitrogen atom; (b) fused to a 5-membered or 6-membered aromatic heterocyclic ring having two nitrogen atoms; (c) fused to a 5-membered aromatic heterocyclic ring having one nitrogen atom together with either one oxygen or one sulfur atom; or (d) fused to a 5-membered aromatic heterocyclic ring having one heteroatom selected from N, O, or S. In one embodiment, the heteroaryl group is optionally substituted.

The term “heterocyclyl” or “heterocyclic” as used herein refers to a three- to fourteen-membered saturated, partially saturated, or unsaturated cycloalkyl group, unless specified otherwise, in which one to four of the ring carbon atoms have been independently replaced with a N, O, or S atom. In some embodiments, the cycloalkyl group is a three- to ten-membered group. In some embodiments, the cycloalkyl group is a three- to seven-membered group. Any suitable ring position of the heterocyclic group may be covalently linked to the defined chemical structure. Exemplary heterocyclic groups include, but are not limited to, azepanyl, azetidinyl, aziridinyl, homopiperazinyl, imidazolidinyl, imidazolinyl, isothiazolyl, morpholinyl, oxadiazolyl, oxazolidinyl, phenanthridinyl, phenanthrolinyl, piperazinyl, piperidinyl, pyranyl, pyrazolidinyl, pyrazolinyl, pyrrolidinyl, pyrrolinyl, quinuclidinyl, tetrahydrofuranyl, thiadiazinyl, thiadiazolyl, thiomorpholinyl, triazinyl, and triazolyl. In one embodiment, the heterocyclic group is optionally substituted.

The term “isolated and purified” as used herein refers to separate from other components of a reaction mixture or a natural source. In certain embodiments, the isolate contains at least about 50%, at least about 55%, at least about 60%, at least about 65%, at least about 70%, at least about 75%, at least about 80%, at least about 85%, at least about 90%, at least about 95%, or at least about 98% of the compound or pharmaceutically acceptable salt of the compound by weight of the isolate.

The term “optionally substituted” as used herein refers to substitution with one or more substituents. In one embodiment, “optionally substituted” refers to substitution with one or more of the following possible substituents (the same or different): —NO₂, -unsubstituted aryl, -unsubstituted heteroaryl, —C(O)—O-unsubstituted alkyl, —O-unsubstituted alkyl, -alkyl-O-unsubstituted alkyl, —O—(C₂-C₄)—O-unsubstituted alkyl, —CN, -halogen, -hydroxy, —N(R′)₂, -trifluoromethyl, -trifluoromethoxy, alkyl substituted with unsubstituted aryl, aryl substituted with unsubstituted alkyl, -alkyl-NR′₂, —(C₁-C₆)-alkyl-OH, -alkyl-O-unsubstituted alkyl, —S-unsubstituted alkyl, —SO₂NR′₂, —SO₂NHR′, —CO₂H, —CON(R′)₂, —O-unsubstituted aryl, —O-unsubstituted heteroaryl, —S-unsubstituted aryl, —S(O)-unsubstituted aryl, —S(O)₂-unsubstituted aryl, -alkyl-O-alkyl-N(R′)₂, -alkyl-aryl-O-alkyl-N(R′)₂, unsubstituted (C₁-C₆)-alkyl, unsubstituted (C₂-C₆)-alkenyl, unsubstituted (C₂-C₆)-alkynyl, unsubstituted cycloalkyl, —O-alkyl-O-unsubstituted alkyl, -S-unsubstituted heteroaryl, —S(O)-unsubstituted heteroaryl, and —S(O)₂-unsubstituted heteroaryl; wherein each R′ is independently hydrogen, (C₁-C₆)-unsubstituted alkyl, unsubstituted aryl, unsubstituted heteroaryl, aryl substituted with unsubstituted alkyl, alkyl substituted with unsubstituted aryl, alkyl substituted with unsubstituted heteroaryl, or heteroaryl substituted with unsubstituted alkyl.

The term “pharmaceutically acceptable salt” as used herein refers to a salt of an acid and a basic nitrogen atom of a compound of the present invention. Exemplary salts include, but are not limited to, sulfate, citrate, acetate, oxalate, chloride, hydrochloride, bromide, hydrobromide, iodide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, succinate, fumarate, maleate, malonate, mandelate, malate, phthalate, and pamoate. The term “pharmaceutically acceptable salt” as used herein also refers to a salt of a compound of the present invention having an acidic functional group, such as a carboxylic acid functional group, and a base. Exemplary bases include, but are not limited to, hydroxide of alkali metals including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like. The term “pharmaceutically acceptable salt” also includes a hydrate of a compound of the present invention.

The following abbreviations as used herein mean: Et₂O means diethyl ether; EtOAc means ethyl acetate; NMR means nuclear magnetic resonance; THF means tetrahydrofuran.

Methods for Synthesizing Compounds of Formula (II)

In one embodiment, the invention provides a method of synthesizing a compound of the formula (II) 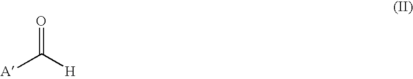
or a pharmaceutically acceptable salt thereof,
wherein A′ is 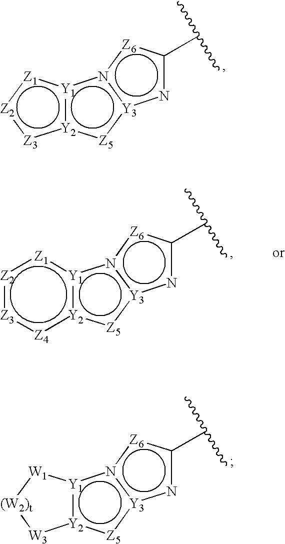
wherein
Z₁-Z₆ are each independently CR₂, N, O, S, or NR₁;
Y₁—Y₃ are each independently C, or N;
W₁—W₃ are each independently CR₄R₄S, S(O), S(O)₂, O, or NR₁;
t is 1, 2, 3, or 4;
R₁ is hydrogen, —(C₁-C₆)-alkyl, -aryl, -heteroaryl, -heterocyclyl, —(C₃-C₇)-cycloalkyl, —(C₂-C₆)-alkenyl, —(C₂-C₆)-alkynyl, —(C₁-C₆)-perfluoroalkyl, —SO₂—(C₁-C₆)-alkyl, —SO₂—(C₁-C₆)-aryl, —C(O)-heteroaryl, —C(O)-aryl, —C(O)—(C₁-C₆)-alkyl, —C(O)—(C₃-C₇)-cycloalkyl, —C(O)-heterocyclyl, -aryl substituted with (C₁-C₆)-alkyl, -heteroaryl substituted with (C₁-C₆)-alkyl, —(C₁-C₆)-alkyl substituted with aryl, —(C₁-C₆)-alkyl substituted with heteroaryl, -heterocyclyl substituted with (C₁-C₆)-alkyl, —(C₂-C₈)-alkenyl substituted with aryl, —C(O)NR₆R₇, —SO₂NR₆R₇, —(C₁-C₆)-alkyl-O—(C₁-C₆)-alkyl-aryl, -aryl substituted with (C₁-C₆)-alkyl-O—(C₁-C₆)-alkyl, -heteroaryl substituted with (C₁-C₆)-alkyl-O—(C₁-C₆)-alkyl, —(C₁-C₆)-alkyl substituted with —O-aryl, —(C₁-C₆)-alkyl substituted with —O-heteroaryl, -aryl substituted with —O-aryl, -heteroaryl substituted with —O-heteroaryl, —(C₁-C₆)-alkyl-aryl-O-aryl, —(C₁-C₆)-alkyl-aryl-O-heteroaryl, —(C₁-C₆)-alkyl-aryl-O—(C₁-C₆)-alkyl-NR₆R₇, —C(O)O—(C₁-C₆)-alkyl, —C(O)O—(C₁-C₆)-aryl, or —C(O)O—(C₁-C₆)-heteroaryl; wherein each -alkyl, -alkenyl, -alkynyl, -cycloalkyl, -aryl, -heterocyclyl, or -heteroaryl is independently optionally substituted with one or more R₁₀;
R₂ is hydrogen, —(C₁-C₆)-alkyl, —(C₂-C₆)-alkenyl, —(C₂-C₆)-alkynyl, -halo, —CN, —NR₆R₇, —O—(C₁-C₆)-alkyl, —OH, -aryl, -heteroaryl, —CO₂R₆, —(C₁-C₆)-alkyl-aryl-O—(C₁-C₆)-alkyl-NR₆R₇, —O-aryl, —O-heteroaryl, —O—(C₃-C₆)-alkynyl, —(C₁-C₆)—O—(C₁-C₆)-alkyl-NR₆R₇, —O—(CH₂)₂—O—, -aryl-O—(C_{-C}₆)-alkyl-NR₆R₇, —(C₁-C₆)-perfluoroalkyl, —S—(C₁-C₆)-alkyl, —S(O)—(C₁-C₆)-alkyl, —S(O)₂—(C₁-C₆)-alkyl, —S-aryl, —S(O)-aryl, —S(O)₂-aryl, —C(O)NR₆R₇, guanidino, cyclic guanidino, -aryl substituted with (C₁-C₆)-alkyl, —(C₁-C₆)-alkyl substituted with aryl, -heteroaryl substituted with (C₁-C₆)-alkyl, —(C₁-C₆)-alkyl substituted with heteroaryl, -heterocyclyl substituted with (C₁-C₆)-alkyl, —(C₂-C₈)-alkenyl substituted with aryl, —SO₂NR₆R₇, —(C₁-C₆)-alkyl-O—(C₁-C₆)-aryl, —(C₁-C₆)-alkyl-O-aryl, —(C₁-C₆)-alkyl-O-heteroaryl, -aryl substituted with —O-aryl, -heteroaryl substituted with —O-aryl,-aryl substituted with —O-heteroaryl, -heteroaryl substituted with —O-heteroaryl, —(C₁-C₆)-alkyl-aryl-O-aryl, —(C₁-C₆)-alkyl substituted with —O-aryl, —(C₁-C₆)-alkyl substituted with —O-heteroaryl, or —(C₁-C₆)-alkyl-aryl-O—(C₁-C₆)-alkyl-NR₆R₇; wherein each -alkyl, -alkenyl, -alkynyl, -cycloalkyl, -aryl, -heterocyclyl, or -heteroaryl is independently optionally substituted with one or more R₁₀;
R₁₀ is —NO₂, -aryl, -heteroaryl, —C(O)O—(C₁-C₆)-alkyl, —O—(C₁-C₆)-alkyl, —(C₁-C₆)-alkyl substituted with —O—(C₁-C₆)-alkyl, —O—(C₂-C₄)-alkyl substituted with —O—(C₁-C₆)-alkyl, —CN, -halo, —OH, —NR₁₁R₁₂, —CF₃, —OCF₃, —(C₁-C₆)-alkyl substituted with aryl, aryl substituted with (C₁-C₆)-alkyl, —(C₁-C₆)-alkyl substituted with —NR₁₁R₁₂, —(C₁-C₆)-alkyl substituted with —OH, —(C₁-C₆)-alkyl substituted with —O—(C₁-C₆)-alkyl, —S—(C₁-C₆)-alkyl, —SO₂NR₁₁R₁₂, —SO₂NHR₁₁, —CO₂H, —C(O)NR₁₀R₁₁, —O-aryl, —O-heteroaryl, —S-aryl, —S(O)-aryl, S(O)₂-aryl, —(C₁-C₆)-alkyl-O—(C₁-C₆)-alkyl-NR₁₁R₁₂, —(C₁-C₆)-alkyl-aryl-O—(C₁-C₆)-alkyl-NR₁₁R₁₂, —(C₁-C₆)-alkyl, —(C₂-C₆)-alkenyl, —(C₂-C₆)-alkynyl, —(C₃-C₇)-cycloalkyl, —O—(C₁-C₆)-alkyl-O—(C₁-C₆)-alkyl, (C₁-C₆)-alkyl substituted with —NR₁₁R₁₂, —S-heteroaryl, —S(O)-heteroaryl, —S(O)₂-heteroaryl;
R₁₁ and R₁₂ are each independently hydrogen, —(C₁-C₆)-alkyl, -aryl, -heteroaryl, -aryl substituted with (C₁-C₆)-alkyl, —(C₁-C₆)-alkyl substituted with aryl, —(C₁-C₆)-alkyl substituted with heteroaryl, -heteroaryl substituted with (C₁-C₆)-alkyl; or R₁₁, R₁₂ and the nitrogen atom to which they are attached are taken together to form a three- to seven-membered saturated heterocyclic ring optionally having one or two additional heteroatoms selected from NR₁, O, S, S(O), or S(O)₂;
R₄ is hydrogen, —(C₁-C₆)-alkyl, —OH, —O—(C₁-C₆)-alkyl, —S—(C₁-C₆)-alkyl, —CO₂R₆, —NR₆R₇, —C(O)NR₆R₇; wherein each alkyl is independently optionally substituted with R₁₀; provided that two R₄ bound to the same carbon atom are not simultaneously —OH; or two R₄ bound to the same carbon atom are taken together to form ═O; or two R₄ and the carbon atom to which they are attached are taken together to form a five- to eight-membered spiro system optionally having up to three heteroatoms selected from NR₁, O, S, S(O), or S(O)₂;
R₆ and R₇ are each independently hydrogen, —(C₁-C₆)-alkyl, -aryl, -heteroaryl, -aryl substituted with (C₁-C₆)-alkyl, —(C₁-C₆)-alkyl substituted with aryl, —(C₁-C₆)-alkyl substituted with heteroaryl, -heteroaryl substituted with (C₁-C₆)-alkyl, wherein each -alkyl, -aryl, or -heteroaryl is optionally substituted with one or more R₁₀; or R₆, R₇ and the nitrogen to which they are attached are taken together to form a three- to seven-membered saturated heterocyclic ring optionally having one or two additional heteroatoms selected from NR₁, O, S, S(O), or S(O)₂;
comprising
a) reacting a compound of the formula (III)
   D-NH₂ (III)
or a pharmaceutically acceptable salt thereof,
wherein D is 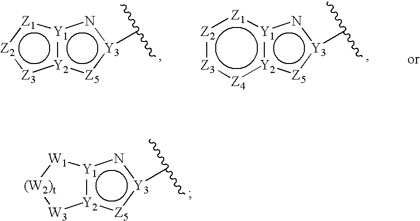
under conditions effective to bring about cyclization, thereby providing an aldehyde of the formula (II).

In one embodiment, the method comprises reacting the compound of the formula (III) with a compound of the formula (VII) 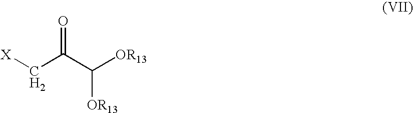
wherein X is a leaving group and R₁₃ is an alkyl or substituted alkyl group, such as (C₁-C₆)-alkyl, or C₇-C₁₅ aralkyl. Exemplary leaving groups include, without limitation, halogen or an organic sulfonyloxy group such as an aryl- or alkyl-sulfonyloxy group, e.g., p-toluenesulfonyl (—OTs), methanesulfonyl (—OMs) or trifluoromethanesulfonyl (—OTr). In one embodiment, X is halogen such as chlorine, bromine or iodine. In one embodiment, X is Br. In one embodiment R₁₃ is methyl. In one embodiment, X is Br and R₁₃ is methyl.

In one embodiment, the compound of the formula (III) is present as its free base.

In another embodiment, the compound of the formula (III) is present as a pharmaceutically acceptable salt.

In one embodiment, the pharmaceutically acceptable salt of the compound of the formula (III) is a hydrochloride salt, a hydroiodide salt, or a hydrobromide salt.

In one embodiment, the method comprises reacting the compound of the formula (III) in the absence of a base.

In another embodiment, the method comprises reacting the compound of the formula (III) in the presence of a base.

In one embodiment, the base is sodium bicarbonate, potassium bicarbonate, pyridine, lutidine, triethylamine, diisopropylamine, or a combination thereof.

In one embodiment, the method comprises reacting the compound of the formula (III) in the presence of a solvent selected from the group consisting of ethanol, 2-propanol, tetrahydrofuran, methylene chloride, acetonitrile, dimethylformamide, ethyl acetate, dichloroethane, dimethylacetamide, N-methylpyrrolidinone, acetone, or a combination thereof.

In one embodiment, the compound of formula (II) is

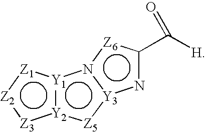

In one embodiment, Z₁, Z₂, Z₃, and Z₆ are each independently CR₂; Z₅ is S; and Y₁, Y₂, Y₃ are each C.

In one embodiment, Z₁, Z₂, and Z₃ are each independently CR₂; Z₅ is S; Z₆ is CR₂ or N; Y₁ and Y₂ are each C; and Y₃ is C or N.

In one embodiment, Z₁ is O, S, or NR₁; Z₂, Z₃, and Z₆ are each independently CR₂ or N; Z₅ is S; Y₁, Y₂, and Y₃ are each C.

In one embodiment, Z₁ is O, S, or NR₁; Z₂, Z₃, and Z₆ are each independently CR₂; Z₅ is S; Y₁, Y₂, and Y₃ are each C.

In one embodiment, Z₂ is O, S, or NR₁; Z₁, Z₃, and Z₆ are each independently CR₂ or N; Z₅ is S; Y₁, Y₂, and Y₃ are each C.

In one embodiment, Z₂ is O, S, or NR₁; Z₁, Z₃, and Z₆ are each independently CR₂; Z₅ is S; Y₁, Y₂, and Y₃ are each C.

In one embodiment, Z₃ is O, S, or NR₁; Z₁, Z₂, and Z₆ are each independently CR₂ or N; Z₅ is S; Y₁, Y₂, and Y₃ are each C.

In one embodiment, Z₃ is O, S, or NR₁; Z₁, Z₂, and Z₆ are each independently CR₂; Z₅ is S; Y₁, Y₂, and Y₃ are each C.

In one embodiment, Z₁ is O, S, or NR₁; Z₂, Z₃, and Z₆ are each independently CR₂ or N; Z₅ is S; Y₂ is N; Y₁ and Y₃ are each C.

In one embodiment, Z₁ is O, S, or NR₁; Z₂, Z₃, and Z₆ are each independently CR₂; Z₅ is S; Y₂ is N; Y₁ and Y₃ are each C.

In one embodiment, Z₂ is O, S, or NR₁; Z₁, Z₃, and Z₆ are each independently CR₂ or N; Z₅ is S; Y₁ is N; Y₂ and Y₃ are each C.

In one embodiment, Z₂ is O, S, or NR₁; Z₁, Z₃, and Z₆ are each independently CR₂; Z₅ is S; Y₁ is N; Y₂ and Y₃ are each C.

In one embodiment, Z₃ is O, S, or NR₁; Z₁, Z₂, and Z₆ are each independently CR₂ or N; Z₅ is S; Y₁ is N; Y₂ and Y₃ are each C.

In one embodiment, Z₃ is O, S, or NR₁; Z₁, Z₂, and Z₆ are each independently CR₂; Z₅ is S; Y₁ is N; Y₂ and Y₃ are each C.

In one embodiment, Z₁ is O, S, or NR₁; Z₂, Z₃, and Z₆ are each independently CR₂ or N; Z₅ is S; Y₁ is N; Y₂ and Y₃ are each C.

In one embodiment, Z₁ is O, S, or NR₁; Z₂, Z₃, and Z₆ are each independently CR₂; Z₅ is S; Y₁ is N; Y₂ and Y₃ are each C.

In one embodiment, each R₂ is independently hydrogen or —(C₁-C₆)-alkyl.

In one embodiment, each R₁ is independently hydrogen or —(C₁-C₆)-alkyl.

In another embodiment, the compound of formula (II) is

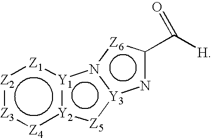

In one embodiment, Z₁, Z₂, Z₃, Z₄, and Z₆ are each independently CR₂; Z₅ is S; and Y₁, Y₂, and Y₃ are each C.

In one embodiment, Z₁, Z₂, Z₃, and Z₄ are each independently CR₂; Z₅ is S; Z₆ is CR₂ or N; Y₁ and Y₂ are each C; and Y₃ is C or N.

In one embodiment, Z₁, Z₂, Z₃, and Z₄ are each independently CR₂; Z₅ is S; Z₆ is CR₂ or N; and Y₁, Y₂, and Y₃ are each C.

In one embodiment, Z₁, Z₂, Z₃, Z₄, and Z₆ are each independently CR₂; Z₅ is S; Y₁ and Y₂ are each C; and Y₃ is C or N.

In one embodiment, Z₁, Z₂, Z₃, Z₄, and Z₆ are each independently CR₂ or N; Z₅ is S; and Y₁, Y₂, and Y₃ are each C.

In one embodiment, each R₂ is independently hydrogen or —(C₁-C₆)-alkyl.

In one embodiment, the compound of formula (II) is

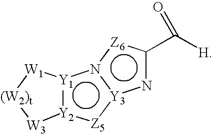

In one embodiment, t is 1; W₁, W₂, and W₃ are each independently CR₄R₄; Z₅ is S; Z₆ is CR₂; and Y₁, Y₂, and Y₃ are each C.

In one embodiment, t is 1 to 3; W₁, W₂, and W₃ are each independently CR₄R₄, O, S, SO, SO₂, or NR₁, provided that no S—S, S—O, or O—O bond formation can occur to form a saturated ring; Z₅ is S; Z₆ is CR₂ or N; and Y₁, Y₂, and Y₃ are each C.

In one embodiment, t is 1 to 3; W₁, W₂, and W₃ are each independently CR₄R₄; Z₅ is S; Z₆ is CR₂ or N; and Y₁, Y₂, and Y₃ are each C.

In one embodiment, t is 1 to 3; W₁, W₂, and W₃ are each independently CR₄R₄, O, S, SO, SO₂, or NR₁, provided that no S—S, S—O, or O—O bond formation can occur to form a saturated ring; Z₅ is S; Z₆ is CR₂; and Y₁, Y₂, and Y₃ are each C.

In one embodiment, t is 1 to 3; W₁, W₂, and W₃ are each independently CR₄R₄; Z₅ is S; Z₆ is CR₂; and Y₁, Y₂, and Y₃ are each C.

In one embodiment, t is 1 to 3; W₁, W₂, and W₃ are each independently CR₄R₄, O, S, SO, SO₂, or NR₁, provided that no S—S, S—O, or O—O bond formation can occur to form a saturated ring; Z₅ is S; Z₆ is CR₂; Y₁ and Y₂ are each C; and Y₃ is N.

In one embodiment, t is 1 to 3; W₁, W₂, and W₃ are each independently CR₄R₄; Z₅ is S; Z₆ is CR₂; Y₁ and Y₂ are each C; and Y₃ is N.

In one embodiment, t is 1.

In one embodiment, each R₁ is independently hydrogen or —(C₁-C₆)-alkyl.

In one embodiment, each R₂ is independently hydrogen or —(C₁-C₆)-alkyl.

In one embodiment, each R₄ is independently hydrogen or —(C₁-C₆)-alkyl.

In one embodiment, the compound or pharmaceutically acceptable salt of the compound of formula (II) is isolated and purified.

In another embodiment, the invention provides a method of synthesizing a compound of the formula (II) 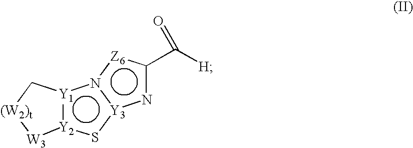
or a pharmaceutically acceptable salt thereof,
wherein
Z₆ is CR₂, N, O, S, or NR₁;
Y₁—Y₃ are each independently C, or N;
W₂—W₃ are each independently CR₄R₄, S(O), S(O)₂, O, or NR₁;
t is 1, 2, 3, or 4;
R₁ is hydrogen, —(C₁-C₆)-alkyl, -aryl, -heteroaryl, -heterocyclyl, —(C₃-C₇)-cycloalkyl, —(C₂-C₆)-alkenyl, —(C₂-C₆)-alkynyl, —(C₁-C₆)-perfluoroalkyl, —SO₂—(C₁-C₆)-alkyl, —SO₂—(C₁-C₆)-aryl, —C(O)-heteroaryl, —C(O)-aryl, —C(O)—(C₁-C₆)-alkyl, —C(O)—(C₃-C₇)-cycloalkyl, —C(O)-heterocyclyl, -aryl substituted with (C₁-C₆)-alkyl, -heteroaryl substituted with (C₁-C₆)-alkyl, —(C₁-C₆)-alkyl substituted with aryl, —(C₁-C₆)-alkyl substituted with heteroaryl, -heterocyclyl substituted with (C₁-C₆)-alkyl, —(C₂-C₈)-alkenyl substituted with aryl, —C(O)NR₆R₇, —SO₂NR₆R₇, —(C₁-C₆)-alkyl-O—(C₁-C₆)-alkyl-aryl, -aryl substituted with (C₁-C₆)-alkyl-O—(C₁-C₆)-alkyl, -heteroaryl substituted with (C₁-C₆)-alkyl-O—(C₁-C₆)-alkyl, —(C₁-C₆)-alkyl substituted with —O-aryl, —(C₁-C₆)-alkyl substituted with —O-heteroaryl, -aryl substituted with —O-aryl, -heteroaryl substituted with —O-heteroaryl, —(C₁-C₆)-alkyl-aryl-O-aryl, —(C₁-C₆)-alkyl-aryl-O-heteroaryl, —(C₁-C₆)-alkyl-aryl-O—(C₁-C₆)-alkyl-NR₆R₇, —C(O)O—(C₁-C₆)-alkyl, —C(O)O—(C₁-C₆)-aryl, or —C(O)O—(C₁-C₆)-heteroaryl; wherein each -alkyl, -alkenyl, -alkynyl, -cycloalkyl, -aryl, -heterocyclyl, or -heteroaryl is independently optionally substituted with one or more R₁₀;
R₂ is hydrogen, —(C₁-C₆)-alkyl, —(C₂-C₆)-alkenyl, —(C₂-C₆)-alkynyl, -halo, —CN, —NR₆R₇, —O—(C₁-C₆)-alkyl, —OH, -aryl, -heteroaryl, —CO₂R₆, —(C₁-C₆)-alkyl-aryl-O—(C₁-C₆)-alkyl-NR₆R₇, —O-aryl, —O-heteroaryl, —O—(C₃-C₆)-alkynyl, —(C₁-C₆)—O—(C₁-C₆)-alkyl-NR₆R₇, —O—(CH₂)₂—O—, -aryl-O—(C₁-C₆)-alkyl-NR₆R₇, —(C₁-C₆)-perfluoroalkyl, —S—(C₁-C₆)-alkyl, —S(O)—(C₁-C₆)-alkyl, —S(O)₂—(C_{-C}₆)-alkyl, —S-aryl, —S(O)-aryl, —S(O)₂-aryl, —C(O)NR₆R₇, guanidino, cyclic guanidino, -aryl substituted with (C₁-C₆)-alkyl, —(C₁-C₆)-alkyl substituted with aryl, -heteroaryl substituted with (C₁-C₆)-alkyl, —(C₁-C₆)-alkyl substituted with heteroaryl, -heterocyclyl substituted with (C₁-C₆)-alkyl, —(C₂-C₈)-alkenyl substituted with aryl, —SO₂NR₆R₇, —(C₁-C₆)-alkyl-O—(C₁-C₆)-aryl, —(C₁-C₆)-alkyl-O-aryl, —(C₁-C₆)-alkyl-O-heteroaryl, -aryl substituted with —O-aryl, -heteroaryl substituted with —O-aryl, -aryl substituted with —O-heteroaryl, -heteroaryl substituted with —O-heteroaryl, —(C₁-C₆)-alkyl-aryl-O-aryl, —(C₁-C₆)-alkyl substituted with —O-aryl, —(C₁-C₆)-alkyl substituted with —O-heteroaryl, or —(C₁-C₆)-alkyl-aryl-O—(C₁-C₆)-alkyl-NR₆R₇; wherein each -alkyl, -alkenyl, -alkynyl, -cycloalkyl, -aryl, -heterocyclyl, or -heteroaryl is independently optionally substituted with one or more R₁₀;
R₁₀ is —NO₂, -aryl, -heteroaryl, —C(O)O—(C₁-C₆)-alkyl, —O—(C₁-C₆)-alkyl, —(C₁-C₆)-alkyl substituted with —O—(C₁-C₆)-alkyl, —O—(C₂-C₄)-alkyl substituted with —O—(C₁-C₆)-alkyl, —CN, -halo, —OH, —NR₁₁R₁₂, —CF₃, —OCF₃, —(C₁-C₆)-alkyl substituted with aryl, aryl substituted with (C₁-C₆)-alkyl, —(C₁-C₆)-alkyl substituted with —NR₁₁R₁₂, —(C₁-C₆)-alkyl substituted with —OH, —(C₁-C₆)-alkyl substituted with —O—(C₁-C₆)-alkyl, —S—(C₁-C₆)-alkyl, —SO₂NR₁₁R₁₂, —SO₂NHR₁₁, —CO₂H, —C(O)NR₁₀R₁₁, —O-aryl, —O-heteroaryl, —S-aryl, —S(O)-aryl, S(O)₂-aryl, —(C₁-C₆)-alkyl-O—(C₁-C₆)-alkyl-NR₁₁R₁₂, —(C₁-C₆)-alkyl-aryl-O—(C₁-C₆)-alkyl-NR₁₁R₁₂, —(C₁-C₆)-alkyl, —(C₂-C₆)-alkenyl, —(C₂-C₆)-alkynyl, —(C₃-C₇-cycloalkyl, —O—(C₁-C₆)-alkyl-O—(C₁-C₆)-alkyl, (C₁-C₆)-alkyl substituted with —NR₁₁R₁₂, —S-heteroaryl, —S(O)-heteroaryl, —S(O)₂-heteroaryl;
R₁₁ and R₁₂ are each independently hydrogen, —(C₁-C₆)-alkyl, -aryl, -heteroaryl, -aryl substituted with (C₁-C₆)-alkyl, —(C₁-C₆)-alkyl substituted with aryl, —(C₁-C₆)-alkyl substituted with heteroaryl, -heteroaryl substituted with (C₁-C₆)-alkyl; or R₁₁, R₁₂ and the nitrogen atom to which they are attached are taken together to form a three- to seven-membered saturated heterocyclic ring optionally having one or two additional heteroatoms selected from NR₁, O, S, S(O), or S(O)₂;
R₄ is hydrogen, —(C₁-C₆)-alkyl, —OH, —O—(C₁-C₆)-alkyl, —S—(C—hd-C₆)-alkyl, —CO₂R₆, —NR₆R₇, —C(O)NR₆R₇; wherein each alkyl is independently optionally substituted with R₁₀; provided that two R₄ bound to the same carbon atom are not simultaneously —OH; or two R₄ bound to the same carbon atom are taken together to form ═O; or two R₄ and the carbon atom to which they are attached are taken together to form a five- to eight-membered spiro system optionally having up to three heteroatoms selected from NR₁, O, S, S(O), or S(O)₂;
R₆ and R₇ are each independently hydrogen, —(C₁-C₆)-alkyl, -aryl, -heteroaryl, -aryl substituted with (C₁-C₆)-alkyl, —(C₁-C₆)-alkyl substituted with aryl, —(C₁-C₆)-alkyl substituted with heteroaryl, -heteroaryl substituted with (C₁-C₆)-alkyl, wherein each -alkyl, -aryl, or -heteroaryl is optionally substituted with one or more R₁₀; or R₆, R₇ and the nitrogen to which they are attached are taken together to form a three- to seven-membered saturated heterocyclic ring optionally having one or two additional heteroatoms selected from NR₁, O, S, S(O), or S(O)₂;
comprising
a) reacting a compound of the formula (VII) 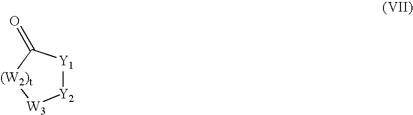
or a pharmaceutically acceptable salt thereof,
under conditions effective to bring about cyclization, thereby providing an amine of the formula (III) 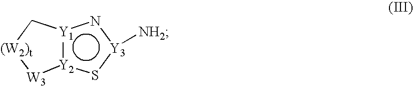
or a pharmaceutically acceptable salt thereof, and
b) reacting a compound of the formula (III) under conditions effective to bring about cyclization, thereby providing an aldehyde of the formula (II).

In one embodiment, the method comprises reacting the compound of the formula (III) with a compound of the formula (VII) 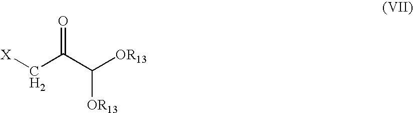
wherein X is a leaving group and R₁₃ is an alkyl or substituted alkyl group, such as (C₁-C₆)-alkyl, or C₇-C₁₅ aralkyl. Exemplary leaving groups include, without limitation, halogen or an organic sulfonyloxy group such as an aryl- or alkyl-sulfonyloxy group, e.g., p-toluenesulfonyl (—OTs), methanesulfonyl (—OMs) or trifluoromethanesulfonyl (—OTr). In one embodiment, X is halogen such as chlorine, bromine or iodine. In one embodiment, X is Br. In one embodiment R₁₃ is methyl. In one embodiment, X is Br and R₁₃ is methyl.

In one embodiment, the compound of the formula (III) is present as its free base.

In another embodiment, the compound of the formula (III) is present as a pharmaceutically acceptable salt.

In one embodiment, the pharmaceutically acceptable salt of the compound of the formula (III) is a hydrochloride salt, a hydroiodide salt, or a hydrobromide salt.

In one embodiment, the method comprises reacting the compound of the formula (III) in the absence of a base.

In another embodiment, the method comprises reacting the compound of the formula (III) in the presence of a base.

In one embodiment, the base is sodium bicarbonate, potassium bicarbonate, pyridine, lutidine, triethylamine, diisopropylethylamine, or a combination thereof.

In one embodiment, the method comprises reacting the compound of the formula (III) in the presence of a solvent selected from the group consisting of ethanol, 2-propanol, tetrahydrofuran, methylene chloride, acetonitrile, dimethylformamide, ethyl acetate, dichloroethane, dimethylacetamide, N-methylpyrrolidinone, acetone, or a combination thereof.

In one embodiment, t is 1; W₂ and W₃ are each independently CR₄R₄; Z₆ is CR₂; and Y₁, Y₂, and Y₃ are each C.

In one embodiment, t is 1 to 3; W₂ and W₃ are each independently CR₄R₄, O, S, SO, SO₂, or NR₁, provided that no S—S, S—O, or O—O bond formation can occur to form a saturated ring; Z₆ is CR₂ or N; and Y₁, Y₂, and Y₃ are each C.

In one embodiment, t is 1 to 3; W₂ and W₃ are each independently CR₄R₄; Z₆ is CR₂ or N; and Y₁, Y₂, and Y₃ are each C.

In one embodiment, t is 1 to 3; W₂ and W₃ are each independently CR₄R₄, O, S, SO, SO₂, or NR₁, provided that no S—S, S—O, or O—O bond formation can occur to form a saturated ring; Z₆ is CR₂; and Y₁, Y₂, and Y₃ are each C.

In one embodiment, t is 1 to 3; W₂ and W₃ are each independently CR₄R₄; Z₆ is CR₂; and Y₁, Y₂, and Y₃ are each C.

In one embodiment, t is 1 to 3; W₂ and W₃ are each independently CR₄R₄, O, S, SO, SO₂, or NR₁, provided that no S—S, S—O, or O—O bond formation can occur to form a saturated ring; Z₆ is CR₂; Y₁ and Y₂ are each C; and Y₃ is N.

In one embodiment, t is 1 to 3; W₂ and W₃ are each independently CR₄R₄; Z₆ is CR₂; Y₁ and Y₂ are each C; and Y₃ is N.

In one embodiment, t is 1.

In one embodiment, each R₁ is independently hydrogen or —(C₁-C₆)-alkyl.

In one embodiment, each R₂ is independently hydrogen or —(C₁-C₆)-alkyl.

In one embodiment, each R₄ is independently hydrogen or —(C₁-C₆)-alkyl.

In one embodiment, the compound of formula (II) is

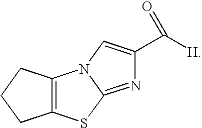

Scheme 1 demonstrates the synthesis of a particular aldehyde of formula (II). As shown in Scheme 1, α-bromination of cyclopentanone gives 2-bromocyclopentanone. Bromination of the cyclopentanone can be achieved, for example, following the procedure outlined below for bromination in Scheme 2. The in situ generated 2-bromocyclopentanone is then subjected to cyclization with thiourea to give the aminothiazole 2, which is isolated as its HBr salt. The salt can be used as is in the following step or can be neutralized to give the free base. Similarly, another aldehyde of formula (II) can be produced by subjecting the 2-bromocyclopentanone to cyclization with urea. When urea is used to effect the cyclization, an oxazole compound is formed (i.e., the sulfur atom of compound 2 is replaced with an oxygen atom).

Cyclization of aminothiazole 2 and bromopyruvaldehyde dimethylacetal 3 can be carried out in a variety of solvents, including, for example, ethanol, 2-propanol, tetrahydrofuran, methylene chloride, and acetonitrile, to provide 1. When the free base of 2 is used, a less than stoichiometric amount of a mild base such as sodium bicarbonate can be added to increase the yield. In some embodiments, the addition of mild base serves to stabilize 2 until the initial alkylation with 3 is complete. In some embodiments, enough acid is generated during the reaction of 2 with 3 to make the reaction mixture acidic, and the change in pH can cause the hydrolysis of the dimethyl acetal group to allow isolation of 1 from the reaction mixture. One of skill in the art will recognize that reactants other than bromopyruvaldehyde dimethylacetal 3 are effective for achieving the desired tricyclic product. For example, the bromine can be replaced with another halogen (e.g., chlorine or iodine) or other leaving group and/or the methoxy groups can be substituted or unsubstituted alkoxy or aralkloxy groups.

In another embodiment, the hydrochloride or hydrobromide salt of 2 can be used in the reaction. In some embodiments, additional mild base can also be used.

Isolation of 1 can be achieved from the reaction mixture by passing a methylene chloride solution of the reaction mixture through a pad of silica gel followed by removal of the solvent. Yields of 1 are typically 30-35% from this reaction.

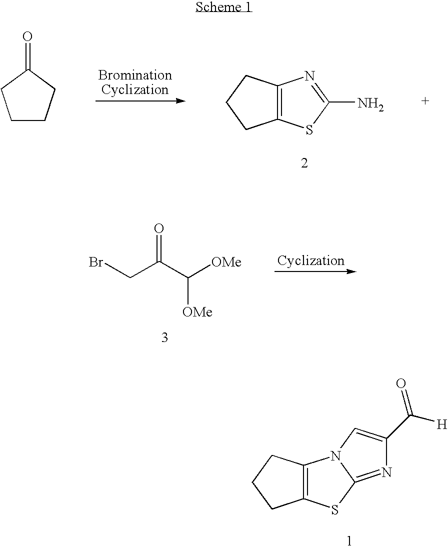

One of skill in the art will recognize that Scheme 1 can be adapted to produce the other compounds and pharmaceutically acceptable salts of compounds of formula (II) according to the present invention.

Methods for Synthesizing Compounds of Formula (VII)

In one embodiment, the invention provides a method of synthesizing a compound of the formula (VII) 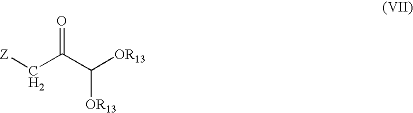
wherein Z is halogen, such as bromine, chlorine, or iodine, and R₁₃ is an alkyl or substituted alkyl group, such as (C₁-C₆)-alkyl, or C₇-C₁₅ aralkyl. In one embodiment, Z is Br. In one embodiment R₁₃ is methyl. In one embodiment, Z is Br and R₁₃ is methyl;

comprising a) reacting a compound of the formula (VIII) 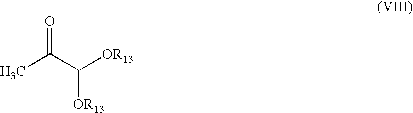
in the presence of a halogenating agent and from about 1% to about 100% (v/v) methanol in acetonitrile, thereby providing a compound of the formula (VII). In one embodiment, Z is Br and the reaction is carried out in the present of a brominating agent.

In one embodiment, the reaction occurs in from about 1% to about 20% (v/v) methanol in acetonitrile.

In another embodiment, the reaction occurs in from about 2% to about 10% (v/v) methanol in acetonitrile.

In one embodiment, the reaction occurs in from about 3% to about 8% (v/v) methanol in acetonitrile.

In another embodiment, the reaction occurs in about 5% (v/v) methanol in acetonitrile.

In one embodiment, the brominating agent is Br₂ or N-bromosuccinimide.

In one embodiment, the compound of the formula (VII) is isolated and purified.

Scheme 2 demonstrates an exemplary synthesis of the compound of formula (VII). α-Bromination of pyruvic aldehyde dimethyl acetal is achieved using 5% (v/v) methanol in acetonitrile to provide 70-80% conversion, along with 5-10% of the dibromopyruvic aldehyde dimethyl acetal and 5-10% of 3-bromo-1,1,2,2-tetramethoxy-propane. The compound 3 can be purified by distillation, or can be used without purification in the cyclization step of Scheme 1. 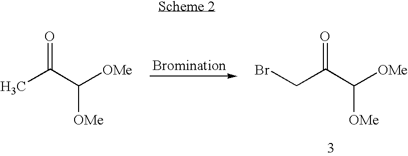
Methods for Synthesizing Compounds of Formula (I)

In one embodiment, the invention provides a method of synthesizing a compound of the formula (I) 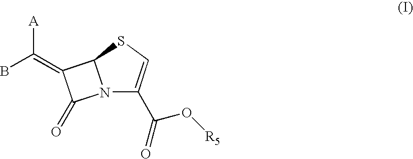
or a pharmaceutically acceptable salt thereof,
wherein
one of A and B is hydrogen and the other of A and B is 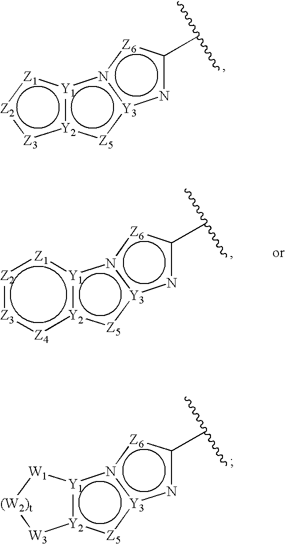
R₅ is hydrogen, —(C₁-C₆)-alkyl, —(C₅-C₆)-cycloalkyl, —CH(R₃)—O—C(O)—(C₁-C₆)-alkyl, benzyhydryl, or p-nitrobenzoyl;
Z₁-Z₆ are each independently CR₂, N, O, S, or NR₁;
Y₁—Y₃ are each independently C, or N;
W₁—W₃ are each independently CR₄R₄, S, S(O), S(O)₂, O, or NR₁;
t is 1, 2, 3, or 4;
R₁ is hydrogen, —(C₁-C₆)-alkyl, -aryl, -heteroaryl, -heterocyclyl, —(C₃-C₇)-cycloalkyl, —(C₂-C₆)-alkenyl, —(C₂-C₆)-alkynyl, —(C₁-C₆)-perfluoroalkyl, —SO₂—(C₁-C₆)-alkyl, —SO₂—(C₁-C₆)-aryl, —C(O)-heteroaryl, —C(O)-aryl, —C(O)—(C₁-C₆)-alkyl, —C(O)—(C₃-C₇)-cycloalkyl, —C(O)-heterocyclyl, -aryl substituted with (C₁-C₆)-alkyl, -heteroaryl substituted with (C₁-C₆)-alkyl, —(C₁-C₆)-alkyl substituted with aryl, —(C₁₃-C₆)-alkyl substituted with heteroaryl, -heterocyclyl substituted with (C₁-C₆)-alkyl, —(C₂-C₈)-alkenyl substituted with aryl, —C(O)NR₆R₇, —SO₂NR₆R₇, —(C₁-C₆)-alkyl-O—(C₁-C₆)-alkyl-aryl, -aryl substituted with (C₁-C₆)-alkyl-O—(C₁-C₆)-alkyl, -heteroaryl substituted with (C₁-C₆)-alkyl-O—(C₁-C₆)-alkyl, —(C₁-C₆)-alkyl substituted with —O-aryl, —(C₁-C₆)-alkyl substituted with —O-heteroaryl, -aryl substituted with —O-aryl, -heteroaryl substituted with —O-heteroaryl, —(C₁-C₆)-alkyl-aryl-O-aryl, —(C₁-C₆)-alkyl-aryl-O-heteroaryl, —(C₁-C₆)-alkyl-aryl-O—(C₁-C₆)-alkyl-NR₆R₇, —C(O)O—(C₁-C₆)-alkyl, —C(O)O—(C₁-C₆)-aryl, or —C(O)O—(C₁-C₆)-heteroaryl; wherein each -alkyl, -alkenyl, -alkynyl, -cycloalkyl, -aryl, -heterocyclyl, or -heteroaryl is independently optionally substituted with one or more R₁₀;
R₂ is hydrogen, —(C₁-C₆)-alkyl, —(C₂-C₆)-alkenyl, —(C₂-C₆)-alkynyl, -halo, —CN, —NR₆R₇, —O—(C₁-C₆)-alkyl, —OH, -aryl, -heteroaryl, —CO₂R₆, —(C₁-C₆)-alkyl-aryl-O—(C₁-C₆)-alkyl-NR₆R₇, —O-aryl, —O-heteroaryl, —O—(C₃-C₆)-alkynyl, —(C₁-C₆)—O—(C₁-C₆)-alkyl-NR₆R₇, —O—(CH₂)₂—O—, -aryl-O—(C₁-C₆)-alkyl-NR₆R₇, —(C₁-C₆)-perfluoroalkyl, —S—(C₁-C₆)-alkyl, —S(O)—(C₁-C₆)-alkyl, —S(O)₂—(C₁-C₆)-alkyl, —S-aryl, —S(O)-aryl, —S(O)₂-aryl, —C(O)NR₆R₇, guanidino, cyclic guanidino, -aryl substituted with (C₁-C₆)-alkyl, —(C₁-C₆)-alkyl substituted with aryl, -heteroaryl substituted with (C₁-C₆)-alkyl, —(C₁-C₆)-alkyl substituted with heteroaryl, -heterocyclyl substituted with (C₁-C₆)-alkyl, —(C₂-C₈)-alkenyl substituted with aryl, —SO₂NR₆R₇, —(C₁-C₆)-alkyl-O—(C₁-C₆)-aryl, —(C₁-C₆)-alkyl-O-aryl, —(C₁-C₆)-alkyl-O-heteroaryl, -aryl substituted with —O-aryl, -heteroaryl substituted with —O-aryl,-aryl substituted with —O-heteroaryl, -heteroaryl substituted with —O-heteroaryl, —(C₁-C₆)-alkyl-aryl-O-aryl, —(C₁-C₆)-alkyl substituted with —O-aryl, —(C₁-C₆)-alkyl substituted with —O-heteroaryl, or —(C₁-C₆)-alkyl-aryl-O—(C₁-C₆)-alkyl-NR₆R₇; wherein each -alkyl, -alkenyl, -alkynyl, -cycloalkyl, -aryl, -heterocyclyl, or -heteroaryl is independently optionally substituted with one or more R₁₀;
R₁₀ is —NO₂, -aryl, -heteroaryl, —C(O)O—(C₁-C₆)-alkyl, —O—(C₁-C₆)-alkyl, —(C₁-C₆)-alkyl substituted with —O—(C₁-C₆)-alkyl, —O—(C₂-C₄)-alkyl substituted with —O—(C₁-C₆)-alkyl, —CN, -halo, —OH, —NR₁₁R₁₂, —CF₃, —OCF₃, —(C₁-C₆)-alkyl substituted with aryl, aryl substituted with (C₁-C₆)-alkyl, —(C₁-C₆)-alkyl substituted with —NR₁₁R₁₂, —(C₁-C₆)-alkyl substituted with —OH, —(C₁-C₆)-alkyl substituted with —O—(C₁-C₆)-alkyl, —S—(C₁-C₆)-alkyl, —SO₂NR₁₁R₁₂, —SO₂NHR₁₁, —CO₂H, —C(O)NR₁₀R₁₁, —O-aryl, —O-heteroaryl, —S-aryl, —S(O)-aryl, S(O)₂-aryl, —(C₁-C₆)-alkyl-O—(C₁C₆)-alkyl-NR₁₁R₁₂, —(C₁-C₆)-alkyl-aryl-O—(C₁-C₆)-alkyl-NR₁₁R₁₂, —(C₁-C₆)-alkyl, —(C₂-C₆)-alkenyl, —(C₂-C₆)-alkynyl, —(C₃-C₇)-cycloalkyl, —O—(C₁-C₆)-alkyl-O—(C₁-C₆)-alkyl, (C₁-C₆)-alkyl substituted with —NR₁₁R₁₂, —S-heteroaryl, —S(O)-heteroaryl, —S(O)₂-heteroaryl;
R₁₁ and R₁₂ are each independently hydrogen, —(C₁-C₆)-alkyl, -aryl, -heteroaryl, -aryl substituted with (C₁-C₆)-alkyl, —(C₁-C₆)-alkyl substituted with aryl, —(C₁-C₆)-alkyl substituted with heteroaryl, -heteroaryl substituted with (C₁-C₆)-alkyl; or R₁₁, R₁₂ and the nitrogen atom to which they are attached are taken together to form a three- to seven-membered saturated heterocyclic ring optionally having one or two additional heteroatoms selected from NR₁, O, S, S(O), or S(O)₂;
R₃ is hydrogen, —(C₁-C₆)-alkyl, —(C₅-C₆)-cycloalkyl, -aryl, or -heteroaryl; wherein each -aryl or -heteroaryl is independently optionally substituted with R₁₀;
R₄ is hydrogen, —(C₁-C₆)-alkyl, —OH, —O—(C₁-C₆)-alkyl, —S—(C₁-C₆)-alkyl, —CO₂R₆, —NR₆R₇, —C(O)NR₆R₇; wherein each alkyl is independently optionally substituted with R₁₀; provided that two R₄ bound to the same carbon atom are not simultaneously —OH; or two R₄ bound to the same carbon atom are taken together to form ═O; or two R₄ and the carbon atom to which they are attached are taken together to form a five- to eight-membered spiro system optionally having up to three heteroatoms selected from NR₁, O, S, S(O), or S(O)₂;
R₆ and R₇ are each independently hydrogen, —(C₁-C₆)-alkyl, -aryl, -heteroaryl, -aryl substituted with (C₁-C₆)-alkyl, —(C₁-C₆)-alkyl substituted with aryl, —(C₁-C₆)-alkyl substituted with heteroaryl, -heteroaryl substituted with (C₁-C₆)-alkyl, wherein each -alkyl, -aryl, or -heteroaryl is optionally substituted with one or more R₁₀; or R₆, R₇ and the nitrogen to which they are attached are taken together to form a three- to seven-membered saturated heterocyclic ring optionally having one or two additional heteroatoms selected from NR₁, O, S, S(O), or S(O)₂;
comprising
a) reacting a compound of the formula (III)
   D-NH₂ (III)
or a pharmaceutically acceptable salt thereof,
wherein D is 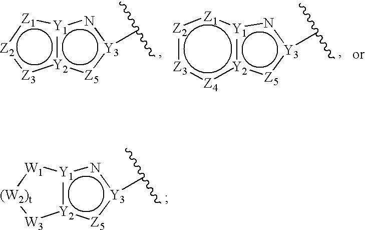
under conditions effective to bring about cyclization, thereby providing an aldehyde of the formula (II): 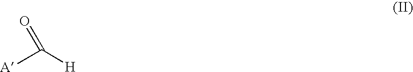
or a pharmaceutically acceptable salt thereof,
wherein A′ is A or B, whichever of A or B is not hydrogen;
b) condensing the aldehyde of formula (II) with a 6-bromo-penem of the formula (IV) 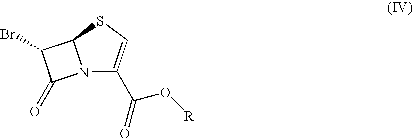
or a pharmaceutically acceptable salt thereof,
where R is a protecting group;
under conditions effective to provide an aldol of the formula (V): 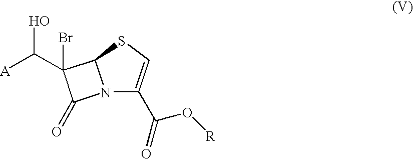
or a pharmaceutically acceptable salt thereof,
c) reacting the aldol of formula (V) under conditions effective to provide a compound of the formula (VI): 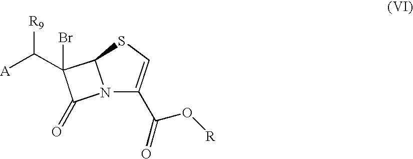
or a pharmaceutically acceptable salt thereof,
wherein R₉ is —X₁ or —OR₈;
X₁ is —Br, —I, or —Cl; and
R₈ is —SO₂-alkyl, —SO₂-aryl, —C(O)-alkyl, or —C(O)-aryl; and
d) subjecting the compound of formula (VI) to conditions effective to bring about reductive elimination, thereby forming a compound of the formula (I).

In one embodiment, the method comprises reacting the compound of the formula (III) with a compound of the formula (VII) 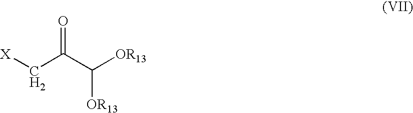
wherein X is a leaving group and R₁₃ is an alkyl or substituted alkyl group, such as (C₁-C₆)-alkyl, or C₇-C₁₅ aralkyl. Exemplary leaving groups include, without limitation, halogen or an organic sulfonyloxy group such as an aryl- or alkyl-sulfonyloxy group, e.g., p-toluenesulfonyl (—OTs), methanesulfonyl (—OMs) or trifluoromethanesulfonyl (—OTr). In one embodiment, X is halogen such as chlorine, bromine or iodine. In one embodiment, X is Br. In one embodiment R₁₃ is methyl. In one embodiment, X is Br and R₁₃ is methyl.

In one embodiment, the compound of the formula (III) is present as its free base.

In another embodiment, the compound of the formula (III) is present as a pharmaceutically acceptable salt.

In one embodiment, the pharmaceutically acceptable salt of the compound of formula (III) is a hydrochloride salt, a hydroiodide salt, or a hydrobromide salt.

In one embodiment, the method comprises reacting the compound of the formula (III) in the absence of a base.

In another embodiment, the method comprises reacting the compound of the formula (III) in the presence of a base.

In one embodiment, the base is sodium bicarbonate, potassium bicarbonate, pyridine, lutidine, triethylamine, diisopropylethylamine, or a combination thereof.

In one embodiment, the method comprises reacting the compound of the formula (III) in the presence of a solvent selected from the group consisting of ethanol, 2-propanol, tetrahydrofuran, methylene chloride, acetonitrile, dimethylformamide, ethyl acetate, dichloroethane, dimethylacetamide, N-methylpyrrolidinone, acetone, or a combination thereof.

In one embodiment, the compound of formula (I) is

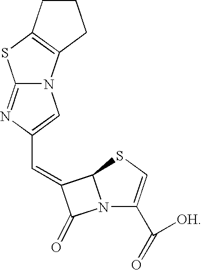

In one embodiment, the compound of formula (II) is

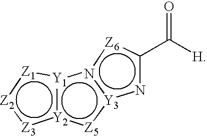

In one embodiment, Z₁, Z₂, Z₃, and Z₆ are each independently CR₂; Z₅ is S; and Y₁, Y₂ and Y₃ are each C.

In one embodiment, Z₁, Z₂, and Z₃ are each independently CR₂; Z₅ is S; Z₆ is CR₂ or N; Y₁, and Y₂ are each C; and Y₃ is C or N.

In one embodiment, Z₁ is O, S, or NR₁; Z₂, Z₃, and Z₆ are each independently CR₂ or N; Z₅ is S; Y₁, Y₂, and Y₃ are each C.

In one embodiment, Z₁ is O, S, or NR₁; Z₂, Z₃, and Z₆ are each independently CR₂; Z₅ is S; Y₁, Y₂, and Y₃ are each C.

In one embodiment, Z₂ is O, S, or NR₁; Z₁, Z₃, and Z₆ are each independently CR₂ or N; Z₅ is S; Y₁, Y₂, and Y₃ are each C.

In one embodiment, Z₂ is O, S, or NR₁; Z₁, Z₃, and Z₆ are each independently CR₂; Z₅ is S; Y₁, Y₂, and Y₃ are each C.

In one embodiment, Z₃ is O, S, or NR₁; Z₁, Z₂, and Z₆ are each independently CR₂ or N; Z₅ is S; Y₁, Y₂, and Y₃ are each C.

In one embodiment, Z₃ is O, S, or NR₁; Z₁, Z₂, and Z₆ are each independently CR₂; Z₅ is S; Y₁, Y₂, and Y₃ are each C.

In one embodiment, Z₁ is O, S, or NR₁; Z₂, Z₃, and Z₆ are each independently CR₂ or N; Z₅ is S; Y₂ is N; Y₁ and Y₃ are each C.

In one embodiment, Z₁ is O, S, or NR₁; Z₂, Z₃, and Z₆ are each independently CR₂; Z₅ is S; Y₂ is N; Y₁ and Y₃ are each C.

In one embodiment, Z₂ is O, S, or NR₁; Z₁, Z₃, and Z₆ are each independently CR₂ or N; Z₅ is S; Y₁ is N; Y₂ and Y₃ are each C.

In one embodiment, Z₂ is O, S, or NR₁; Z₁, Z₃, and Z₆ are each independently CR₂; Z₅ is S; Y₁ is N; Y₂ and Y₃ are each C.

In one embodiment, Z₃ is O, S, or NR₁; Z₁, Z₂, and Z₆ are each independently CR₂ or N; Z₅ is S; Y₁ is N; Y₂ and Y₃ are each C.

In one embodiment, Z₃ is O, S, or NR₁; Z₁, Z₂, and Z₆ are each independently CR₂; Z₅ is S; Y₁ is N; Y₂ and Y₃ are each C.

In one embodiment, Z₁ is O, S, or NR₁; Z₂, Z₃, and Z₆ are each independently CR₂ or N; Z₅ is S; Y₁ is N; Y₂ and Y₃ are each C.

In one embodiment, Z₁ is O, S, or NR₁; Z₂, Z₃, and Z₆ are each independently CR₂; Z₅ is S; Y₁ is N; Y₂ and Y₃ are each C.

In one embodiment, each R₂ is independently hydrogen or —(C₁-C₆)-alkyl.

In one embodiment, each R₁ is independently hydrogen or —(C₁-C₆)-alkyl.

In another embodiment, the compound of formula (II) is

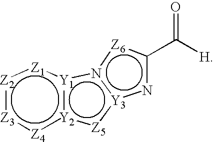

In one embodiment, Z₁, Z₂, Z₃, Z₄, and Z₆ are each independently CR₂; Z₅ is S; and Y₁, Y₂, and Y₃ are each C.

In one embodiment, Z₁, Z₂, Z₃, and Z₄ are each independently CR₂; Z₅ is S; Z₆ is CR₂ or N; Y₁ and Y₂ are each C; and Y₃ is C or N.

In one embodiment, Z₁, Z₂, Z₃, and Z₄ are each independently CR₂; Z₅ is S; Z₆ is CR₂ or N; and Y₁, Y₂, and Y₃ are each C.

In one embodiment, Z₁, Z₂, Z₃, Z₄, and Z₆ are each independently CR₂; Z₅ is S; Y₁ and Y₂ are each C; and Y₃ is C or N.

In one embodiment, Z₁, Z₂, Z₃, Z₄, and Z₆ are each independently CR₂ or N; Z₅ is S; and Y₁, Y₂, and Y₃ are each C.

In one embodiment, each R₂ is independently hydrogen or —(C₁-C₆)-alkyl.

In one embodiment, the compound of formula (II) is

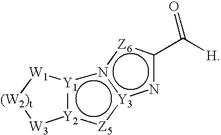

In one embodiment, t is 1; W₁, W₂, and W₃ are each independently CR₄R₄; Z₅ is S; Z₆ is CR₂; and Y₁, Y₂, and Y₃ are each C.

In one embodiment, t is 1 to 3; W₁, W₂, and W₃ are each independently CR₄R₄, O, S, SO, SO₂, or NR₁, provided that no S—S, S—O, or O—O bond formation can occur to form a saturated ring; Z₅ is S; Z₆ is CR₂ or N; and Y₁, Y₂, and Y₃ are each C.

In one embodiment, t is 1 to 3; W₁, W₂, and W₃ are each independently CR₄R₄; Z₅ is S; Z₆ is CR₂ or N; and Y₁, Y₂, and Y₃ are each C.

In one embodiment, t is 1 to 3; W₁, W₂, and W₃ are each independently CR₄R₄, O, S, SO, SO₂, or NR₁, provided that no S—S, S—O, or O—O bond formation can occur to form a saturated ring; Z₅ is S; Z₆ is CR₂; and Y₁, Y₂, and Y₃ are each C.

In one embodiment, t is 1 to 3; W₁, W₂, and W₃ are each independently CR₄R₄; Z₅ is S; Z₆ is CR₂; and Y₁, Y₂, and Y₃ are each C.

In one embodiment, t is 1 to 3; W₁, W₂, and W₃ are each independently CR₄R₄, O, S, SO, SO₂, or NR₁, provided that no S—S, S—O, or O—O bond formation can occur to form a saturated ring; Z₅ is S; Z₆ is CR₂; Y₁ and Y₂ are each C; and Y₃ is N.

In one embodiment, t is 1 to 3; W₁, W₂, and W₃ are each independently CR₄R₄; Z₅ is S; Z₆ is CR₂; Y₁ and Y₂ are each C; and Y₃ is N.

In one embodiment, t is 1.

In one embodiment, each R₁ is independently hydrogen or —(C₁-C₆)-alkyl.

In one embodiment, each R₂ is independently hydrogen or —(C₁-C₆)-alkyl.

In one embodiment, each R₄ is independently hydrogen or —(C₁-C₆)-alkyl.

In one embodiment, the protecting group is p-nitrobenzyl, benzyl, para-methoxy benzyl, benzylhydrol, or trityl.

Exemplary conditions for achieving steps b), c), and d) are as set forth in United States Patent Application Publication No. 2004/0132708 A1, which is incorporated by reference herein in its entirety.

In another embodiment, the invention provides a compound of the formula (I): 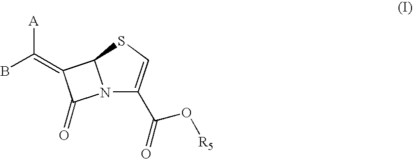
or a pharmaceutically acceptable salt thereof,
wherein
one of A and B is hydrogen and the other of A and B is 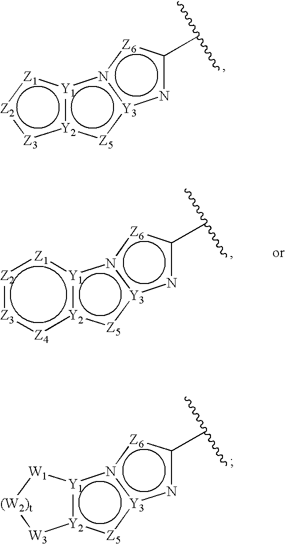
wherein
R₅ is hydrogen, —(C₁-C₆)-alkyl, —(C₅-C₆)-cycloalkyl, or —CH(R₃)—O—C(O)—(C₁-C₆)-alkyl;
Z₁-Z₆ are each independently CR₂, N, O, S, or NR₁;
Y₁—Y₃ are each independently C, N;
W₁—W₃ are each independently CR₄R₄, S, S(O), S(O)₂, O, NR₁;
t is 1, 2, 3, or 4;
R₁ is hydrogen, —(C₁-C₆)-alkyl, -aryl, -heteroaryl, -heterocyclyl, —(C₃-C₇)-cycloalkyl, —(C₂-C₆)-alkenyl, —(C₂-C₆)-alkynyl, —(C₁-C₆)-perfluoroalkyl, —SO₂—(C₁-C₆)-alkyl, —SO₂—(C₁-C₆)-aryl, —C(O)-heteroaryl, —C(O)-aryl, —C(O)—(C₁-C₆)-alkyl, —C(O)—(C₃-C₇)-cycloalkyl, —C(O)-heterocyclyl, -aryl substituted with (C₁-C₆)-alkyl, -heteroaryl substituted with (C₁-C₆)-alkyl, —(C₁-C₆)-alkyl substituted with aryl, —(C₁-C₆)-alkyl substituted with heteroaryl, -heterocyclyl substituted with (C₁-C₆)-alkyl, —(C₂-C₈)-alkenyl substituted with aryl, —C(O)NR₆R₇, —SO₂NR₆R₇, —(C₁-C₆)-alkyl-O—(C₁-C₆)-alkyl-aryl, -aryl substituted with (C₁-C₆)-alkyl-O—(C₁-C₆)-alkyl, -heteroaryl substituted with (C₁-C₆)-alkyl-O—(C₁-C₆)-alkyl, —(C₁-C₆)-alkyl substituted with —O-aryl, —(C₁-C₆)-alkyl substituted with —O-heteroaryl, -aryl substituted with —O-aryl, -heteroaryl substituted with —O-heteroaryl, —(C₁-C₆)-alkyl-aryl-O-aryl, —(C₁-C₆)-alkyl-aryl-O-heteroaryl, —(C₁-C₆)-alkyl-aryl-O—(C₁-C₆)-alkyl-NR₆R₇, —C(O)O—(C₁-C₆)-alkyl, —C(O)O—(C₁-C₆)-aryl, or —C(O)O—(C₁-C₆)-heteroaryl; wherein each -alkyl, -alkenyl, -alkynyl, -cycloalkyl, -aryl, -heterocyclyl, or -heteroaryl is independently optionally substituted with one or more R₁₀;
R₂ is hydrogen, —(C₁-C₆)-alkyl, —(C₂-C₆)-alkenyl, —(C₂-C₆)-alkynyl, -halo, —CN, —NR₆R₇, —O—(C₁-C₆)-alkyl, —OH, -aryl, -heteroaryl, —CO₂R₆, —(C₁-C₆)-alkyl-aryl-O—(C₁-C₆)-alkyl-NR₆R₇, —O-aryl, —O-heteroaryl, —O—(C₃-C₆)-alkynyl, —(C₁-C₆)—O—(C₁-C₆)-alkyl-NR₆R₇, —O—(CH₂)₂—O—, -aryl-O—(C₁-C₆)-alkyl-NR₆R₇, —(C₁-C₆)-perfluoroalkyl, —S—(C₁-C₆)-alkyl, —S(O)—(C₁-C₆)-alkyl, —S(O)₂—(C₁-C₆)-alkyl, —S-aryl, —S(O)-aryl, —S(O)₂-aryl, —C(O)NR₆R₇, guanidino, cyclic guanidino, -aryl substituted with (C₁-C₆)-alkyl, —(C₁-C₆)-alkyl substituted with aryl, -heteroaryl substituted with (C₁-C₆)-alkyl, —(C₁-C₆)-alkyl substituted with heteroaryl, -heterocyclyl substituted with (C₁-C₆)-alkyl, —(C₂-C₈)-alkenyl substituted with aryl, —SO₂NR₆R₇, —(C₁-C₆)-alkyl-O—(C₁-C₆)-aryl, —(C₁-C₆)-alkyl-O-aryl, —(C₁-C₆)-alkyl-O-heteroaryl, -aryl substituted with —O-aryl, -heteroaryl substituted with —O-aryl,-aryl substituted with —O-heteroaryl, -heteroaryl substituted with —O-heteroaryl, —(C₁-C₆)-alkyl-aryl-O-aryl, —(C₁-C₆)-alkyl substituted with —O-aryl, —(C₁-C₆)-alkyl substituted with —O-heteroaryl, or —(C₁-C₆)-alkyl-aryl-O—(C₁-C₆)-alkyl-NR₆R₇; wherein each -alkyl, -alkenyl,-alkynyl, -cycloalkyl, -aryl, -heterocyclyl, or -heteroaryl is independently optionally substituted with one or more Rio;
R₁₀ is —NO₂, -aryl, -heteroaryl, —C(O)O—(C₁-C₆)-alkyl, —O—(C₁-C₆)-alkyl, —(C₁-C₆)-alkyl substituted with —O—(C₁-C₆)-alkyl, —O—(C₂-C₄)-alkyl substituted with —O—(C₁-C₆)-alkyl, —CN, -halo, —OH, —NR₁₁R₁₂, —CF₃, —OCF₃, —(C₁-C₆)-alkyl substituted with aryl, aryl substituted with (C₁-C₆)-alkyl, —(C₁-C₆)-alkyl substituted with —NR₁₁R₁₂, —(C₁-C₆)-alkyl substituted with —OH, —(C₁-C₆)-alkyl substituted with —O—(C₁-C₆)-alkyl, —S—(C₁-C₆)-alkyl, —SO₂NR₁₁R₁₂, —SO₂NHR₁₁, —CO₂H, —C(O)NR₁₀R₁₁, —O-aryl, —O-heteroaryl, —S-aryl, —S(O)-aryl, S(O)₂-aryl, —(C₁-C₆)-alkyl-O—(C₁-C₆)-alkyl-NR₁₁R₁₂, —(C₁-C₆)-alkyl-aryl-O—(C₁-C₆)-alkyl-NR₁₁R₁₂, —(C₁-C₆)-alkyl, —(C₂-C₆)-alkenyl, —(C₂-C₆)-alkynyl, —(C₃-C₇)-cycloalkyl, —O—(C₁-C₆)-alkyl-O—(C₁-C₆)-alkyl, (C₁-C₆)-alkyl substituted with —NR₁₁R₁₂, —S-heteroaryl, —S(O)-heteroaryl, —S(O)₂-heteroaryl;
R₁₁ and R₁₂ are each independently hydrogen, —(C₁-C₆)-alkyl, -aryl, -heteroaryl, -aryl substituted with (C₁-C₆)-alkyl, —(C₁-C₆)-alkyl substituted with aryl, —(C₁-C₆)-alkyl substituted with heteroaryl, -heteroaryl substituted with (C₁-C₆)-alkyl; or R₁₁, R₁₂ and the nitrogen atom to which they are attached are taken together to form a three- to seven-membered saturated heterocyclic ring optionally having one or two additional heteroatoms selected from NR₁, O, S, S(O), or S(O)₂;
R₃ is hydrogen, —(C₁-C₆)-alkyl, —(C₅-C₆)-cycloalkyl, -aryl, or -heteroaryl; wherein each -aryl or -heteroaryl is independently optionally substituted with R₁₀;
R₄ is hydrogen, —(C₁-C₆)-alkyl, —OH, —O—(C₁-C₆)-alkyl, —S—(C₁-C₆)-alkyl, —CO₂R₆, —NR₆R₇, —C(O)NR₆R₇; wherein each alkyl is independently optionally substituted with R₁₀; provided that two R₄ bound to the same carbon atom are not simultaneously —OH; or two R₄ bound to the same carbon atom are taken together to form ═O; or two R₄ and the carbon atom to which they are attached are taken together to form a five- to eight-membered spiro system optionally having up to three heteroatoms selected from NR₁, O, S, S(O), or S(O)₂;
R₆ and R₇ are each independently hydrogen, —(C₁-C₆)-alkyl, -aryl, -heteroaryl, -aryl substituted with (C₁-C₆)-alkyl, —(C₁-C₆)-alkyl substituted with aryl, —(C₁-C₆)-alkyl substituted with heteroaryl, -heteroaryl substituted with (C₁-C₆)-alkyl, wherein each -alkyl, -aryl, or -heteroaryl is optionally substituted with one or more R₁₀; or R₆, R₇ and the nitrogen to which they are attached are taken together to form a three- to seven-membered saturated heterocyclic ring optionally having one or two additional heteroatoms selected from NR₁, O, S, S(O), or S(O)₂;
prepared by the method comprising:
a) reacting a compound of the formula (III)
   D-NH₂ (III)
or a pharmaceutically acceptable salt thereof,
wherein D is 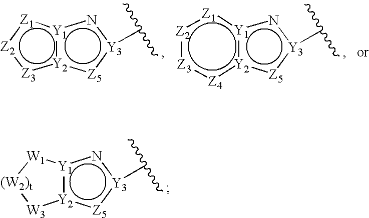
under conditions effective to bring about cyclization, thereby providing an aldehyde of the formula (II): 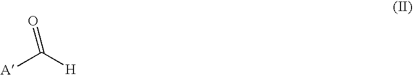
or a pharmaceutically acceptable salt thereof,
wherein A′ is A or B, whichever of A or B is not hydrogen;
b) condensing the aldehyde of formula (II) with a 6-bromo-penem of the formula (IV) 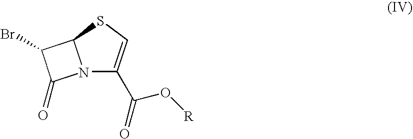
or a pharmaceutically acceptable salt thereof,
where R is a protecting group;
under conditions effective to provide an aldol of the formula (V): 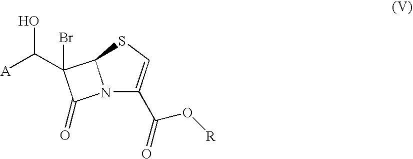
or a pharmaceutically acceptable salt thereof,
c) reacting the aldol of formula (V) under conditions effective to provide a compound of the formula (VI): 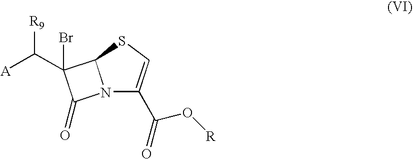
wherein R₉ is —X₁ or —OR₈;
X₁ is —Br, —I, or —Cl; and
R₈ is —SO₂-alkyl, —SO₂-aryl, —C(O)-alkyl, or —C(O)-aryl;
d) subjecting the compound of formula (VI) to conditions effective to bring about reductive elimination, thereby forming a compound of the formula (I).

In one embodiment, the method comprises reacting the compound of the formula (III) with a compound of the formula (VII)

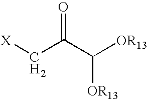

### (VII)

wherein X is a leaving group and R₁₃ is an alkyl or substituted alkyl group, such as (C₁-C₆)-alkyl, or C₇-C₁₅ aralkyl. Exemplary leaving groups include, without limitation, halogen or an organic sulfonyloxy group such as an aryl- or alkyl-sulfonyloxy group, e.g., p-toluenesulfonyl (—OTs), methanesulfonyl (—OMs) or trifluoromethanesulfonyl (—OTr). In one embodiment, X is halogen such as chlorine, bromine or iodine. In one embodiment, X is Br. In one embodiment R₁₃ is methyl. In one embodiment, X is Br and R₁₃ is methyl.

In one embodiment, the compound of the formula (III) is present as its free base.

In another embodiment, the compound of the formula (III) is present as a pharmaceutically acceptable salt.

In one embodiment, the pharmaceutically acceptable salt of the compound of formula (III) is a hydrochloride salt, a hydroiodide salt, or a hydrobromide salt.

In one embodiment, the method comprises reacting the compound of the formula (III) in the absence of a base.

In another embodiment, the method comprises reacting the compound of the formula (III) in the presence of a base.

In one embodiment, the base is sodium bicarbonate, potassium bicarbonate, pyridine, lutidine, triethylamine, diisopropylethylamine, or a combination thereof.

In one embodiment, the method comprises reacting the compound of the formula (III) in the presence of a solvent selected from the group consisting of ethanol, 2-propanol, tetrahydrofuran, methylene chloride, acetonitrile, dimethylformamide, ethyl acetate, dichloroethane, dimethylacetamide, N-methylpyrrolidinone, acetone, or a combination thereof.

In one embodiment, the compound of formula (I) is

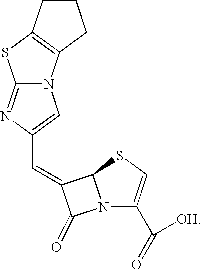

In one embodiment, the compound of formula (II) is

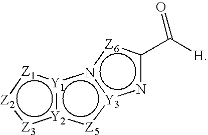

In one embodiment, Z₁, Z₂, Z₃, and Z₆ are each independently CR₂; Z₅ is S; and Y₁, Y₂, and Y₃ are each C.

In one embodiment, Z₁, Z₂, and Z₃ are each independently CR₂; Z₅ is S; Z₆ is CR₂ or N; Y₁ and Y₂ are each C; and Y₃ is C or N.

In one embodiment, Z₁ is O, S, or NR₁; Z₂, Z₃, and Z₆ are each independently CR₂ or N; is Z₅ is S; Y₁, Y₂, and Y₃ are each C.

In one embodiment, Z₁ is O, S, or NR₁; Z₂, Z₃, and Z₆ are each independently CR₂; Z₅ is S; Y₁, Y₂, and Y₃ are each C.

In one embodiment, Z₂ is O, S, or NR₁; Z₁, Z₃, and Z₆ are each independently CR₂ or N; Z₅ is S; Y₁, Y₂, and Y₃ are each C.

In one embodiment, Z₂ is O, S, or NR₁; Z₁, Z₃, and Z₆ are each independently CR₂; Z₅ is S; Y₁, Y₂, and Y₃ are each C.

In one embodiment, Z₃ is O, S, or NR₁; Z₁, Z₂, and Z₆ are each independently CR₂ or N; Z₅ is S; Y₁, Y₂, and Y₃ are each C.

In one embodiment, Z₃ is O, S, or NR₁; Z₁, Z₂, and Z₆ are each independently CR₂; Z₅ is S; Y₁, Y₂, and Y₃ are each C.

In one embodiment, Z₁ is O, S, or NR₁; Z₂, Z₃, and Z₆ are each independently CR₂ or N; Z₅ is S; Y₂ is N; Y₁ and Y₃ are each C.

In one embodiment, Z₁ is O, S, or NR₁; Z₂, Z₃, and Z₆ are each independently CR₂; Z₅ is S; Y₂ is N; Y₁ and Y₃ are each C.

In one embodiment, Z₂ is O, S, or NR₁; Z₁, Z₃, and Z₆ are each independently CR₂ or N; Z₅ is S; Y₁ is N; Y₂ and Y₃ are each C.

In one embodiment, Z₂ is O, S, or NR₁; Z₁, Z₃, and Z₆ are each independently CR₂; Z₅ is S; Y₁ is N; Y₂ and Y₃ are each C.

In one embodiment, Z₃ is O, S, or NR₁; Z₁, Z₂, and Z₆ are each independently CR₂ or N; Z₅ is S; Y₁ is N; Y₂ and Y₃ are each C.

In one embodiment, Z₃ is O, S, or NR₁; Z₁, Z₂, and Z₆ are each independently CR₂; Z₅ is S; Y₁ is N; Y₂ and Y₃ are each C.

In one embodiment, Z₁ is O, S, or NR₁; Z₂, Z₃, and Z₆ are each independently CR₂ or N; Z₅ is S; Y₁ is N; Y₂ and Y₃ are each C.

In one embodiment, Z₁ is O, S, or NR₁; Z₂, Z₃, and Z₆ are each independently CR₂; Z₅ is S; Y₁ is N; Y₂ and Y₃ are each C.

In one embodiment, each R₂ is independently hydrogen or —(C₁-C₆)-alkyl.

In one embodiment, each R₁ is independently hydrogen or —(C₁-C₆)-alkyl.

In another embodiment, the compound of formula (II) is

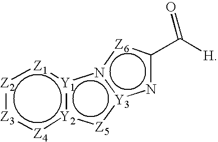

In one embodiment, Z₁, Z₂, Z₃, Z₄, and Z₆ are each independently CR₂; Z₅ is S; and Y₁, Y₂, and Y₃ are each C.

In one embodiment, Z₁, Z₂, Z₃, and Z₄ are each independently CR₂; Z₅ is S; Z₆ is CR₂ or N; Y₁ and Y₂ are each C; and Y₃ is C or N.

In one embodiment, Z₁, Z₂, Z₃, and Z₄ are each independently CR₂; Z₅ is S; Z₆ is CR₂ or N; and Y₁, Y₂, and Y₃ are each C.

In one embodiment, Z₁, Z₂, Z₃, Z₄, and Z₆ are each independently CR₂; Z₅ is S; Y₁ and Y₂ are each C; and Y₃ is C or N.

In one embodiment, Z₁, Z₂, Z₃, Z₄, and Z₆ are each independently CR₂ or N; Z₅ is S; and Y₁, Y₂, and Y₃ are each C.

In one embodiment, each R₂ is independently hydrogen or —(C₁-C₆)-alkyl.

In one embodiment, the compound of formula (II) is

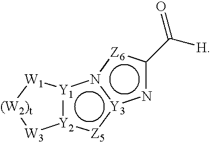

In one embodiment, t is 1; W₁, W₂, and W₃ are each independently CR₄R₄; Z₅ is S; Z₆ is CR₂; and Y₁, Y₂, and Y₃ are each C.

In one embodiment, t is 1 to 3; W₁, W₂, and W₃ are each independently CR₄R₄, O, S, SO, SO₂, or NR₁, provided that no S—S, S—O, or O—O bond formation can occur to form a saturated ring; Z₅ is S; Z₆ is CR₂ or N; and Y₁, Y₂, and Y₃ are each C.

In one embodiment, t is 1 to 3; W₁, W₂, and W₃ are each independently CR₄R₄; Z₅ is S; Z₆ is CR₂ or N; and Y₁, Y₂, and Y₃ are each C.

In one embodiment, t is 1 to 3; W₁, W₂, and W₃ are each independently CR₄R₄, O, S, SO, SO₂, or NR₁, provided that no S—S, S—O, or O—O bond formation can occur to form a saturated ring; Z₅ is S; Z₆ is CR₂; and Y₁, Y₂, and Y₃ are each C.

In one embodiment, t is 1 to 3; W₁, W₂, and W₃ are each independently CR₄R₄; Z₅ is S; Z₆ is CR₂; and Y₁, Y₂, and Y₃ are each C.

In one embodiment, t is 1 to 3; W₁, W₂, and W₃ are each independently CR₄R₄, O, S, SO, SO₂, or NR₁, provided that no S—S, S—O, or O—O bond formation can occur to form a saturated ring; Z₅ is S; Z₆ is CR₂; Y₁ and Y₂ are each C; and Y₃ is N.

In one embodiment, t is 1 to 3; W₁, W₂, and W₃ are each independently CR₄R₄; Z₅ is S; Z₆ is CR₂; Y₁ and Y₂ are each C; and Y₃ is N.

In one embodiment, t is 1.

In one embodiment, each R₁ is independently hydrogen or —(C₁-C₆)-alkyl.

In one embodiment, each R₂ is independently hydrogen or —(C₁-C₆)-alkyl.

In one embodiment, each R₄ is independently hydrogen or —(C₁-C₆)-alkyl.

In one embodiment, the protecting group is p-nitrobenzyl, benzyl, para-methoxy benzyl, benzylhydrol, or trityl.

Exemplary conditions for achieving steps b), c), and d) are as set forth in United States Patent Application Publication No. 2004/0132708 A1, which is incorporated by reference herein in its entirety.

In one embodiment, the compound or pharmaceutically acceptable salt of the compound of formula (I) is isolated and purified.

### EXAMPLES

### Example 1

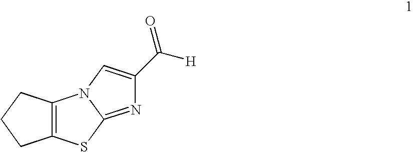

6,7-Dihydro-5H-cyclopenta[d]imidazo[2,1-b]thiazole-2-carbaldehyde (1): A solution of 5,6-dihydro-4H-cyclopentathiazol-2-ylamine (2) (200 g free base, 1.43 mol) in 2-propanol (1 L) was added over 30 minutes to a stirred, cooled (0-5° C.) suspension of bromopyruvaldehyde dimethylacetal 3 (530 g, 2.75 mol), sodium bicarbonate (55 g, 0.66 mol), and 2-propanol (0.30 L). The mixture was allowed to stir for 16 h. The mixture was warmed to 40° C. for 8 h, and was concentrated under reduced pressure to provide a red residue. The red residue was added to methyl t-butyl ether (2.0 L). The mixture was stirred for a minimum of 1 h. The solid was collected by filtration and washed with Et₂O. The dried solid (550 g) was dissolved in methylene chloride (2.75 L). The methylene chloride mixture was passed through a pad of silica gel (1.65 kg). The pad was washed with methylene chloride and 10% EtOAc/methylene chloride. The filtrate was concentrated to a yellow solid, which was triturated with Et₂O and collected by filtration to give 81 g (30%) of the title compound. ¹H NMR (CDCl₃) δ 9.93 (1H, s), 7.91 (1H, s), 2.99-2.89 (4H, m), 2.62-2.57 (2H, m).

### Example 2

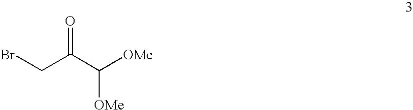

3-Bromo-1,1-dimethoxy-propan-2-one (3): Pyruvaldehyde dimethylacetal (1,1-dimethoxy-propan-2-one ) (400 g, 3.39 mol) and 5% (v/v) methanol/acetonitrile (2.0 L) were combined and cooled to <5° C. with stirring. A solution of bromine (542 g, 3.39 mol) and acetonitrile (300 mL) was prepared. A 10 mL portion of the bromine/acetonitrile solution was added to the pyruvaldehyde dimethylacetal solution and the mixture was stirred until the solution was decolorized (about 30 minutes). The remainder of the bromine/acetonitrile solution was added to the reaction mixture over 6-8 h while maintaining an internal temperature of <10° C. The mixture was stirred at room temperature overnight. Sodium bicarbonate (300 g, 3.6 mol) was added and the mixture was stirred for a minimum of 1 h. The mixture was clarified by filtration and the filtrate was concentrated under reduced pressure using a bath temperature of <35° C. Methyl t-butyl ether (300 mL) and heptane (150 mL) were added to the residue. The mixture was washed with a solution of sodium bicarbonate (60 g, 0.71 mol) and water (600 mL). The organic layer was dried (sodium sulfate) and passed through Magnesol (200 g) and the pad was washed with methyl t-butyl ether (350 mL). The solution was concentrated to give the title compound (475 g, 71%). ¹H NMR (CDCl₃) δ 4.73 (1H, s), 4.21 (2H, s), 3.45 (6H, s).

### Example 3

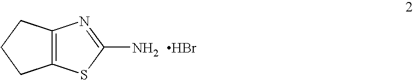

5,6-Dihydro-4H-cyclopentathiazol-2-ylamine (2), HBr salt: A solution of bromine (256 g, 1.6 mol) and acetonitrile (144 mL) was added at a rate of 11 mL/min to a cooled (ice/water bath), stirred solution of cyclopentanone (150 g, 1.78 mol), methanol (75 mL), and acetonitrile (1.425 L). The reaction mixture was allowed to stir at room temperature overnight. The reaction mixture was clarified by filtration and concentrated under reduced pressure to 542 g. The residue was diluted with 2-propanol (750 mL) and thiourea (102 g, 1.34 mol) was added. The mixture was warmed to reflux (72° C.) for 2 h. The mixture was allowed to stir for 16 h at room temperature to complete precipitation of the product, which was then collected by filtration and dried in vacuo to give the title compound as the hydrobromide salt (90.1 g, 31%). ¹H NMR (CD₃OD) δ 4.88 (2H, s), 2.75-2.70 (2H, m), 2.60-2.55 (2H, m), 2.39-2.33 (2H, m).

### Example 4

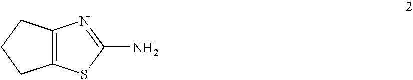

5,6-Dihydro-4H-cyclopentathiazol-2-ylamine (2), free base: The hydrochloride salt of 2 (16.5 g, 93 mmol) (for synthesis of HCl salt, see, e.g., Erlenmeyer and Scheonauer, Helv. Chim. Acta, vol. 24, p. 172E and 175 (1941) and Huenig et al., Chem. Ber., vol. 93, p. 1518-1525 (1960)) was added to a mixture of aqueous sodium bicarbonate (16.5 g, 250 mL) and 25% THF/EtOAc (160 mL, v/v). The mixture was stirred at room temperature for 15 min. The mixture was clarified by filtration through a pad of celite. The organic layer was separated, dried (MgSO₄), and concentrated in vacuo to 47 g. Toluene (100 mL) was added and the mixture was concentrated in vacuo to 45 g. The mixture was diluted with heptane (40 mL), cooled, and the product was collected by filtration and dried to give the title compound as the free base (10.75 g, 85%). ¹H NMR (CD₃OD) δ 4.88 (2H, s), 2.75-2.70 (2H, m), 2.60-2.55 (2H, m), 2.39-2.33 (2H, m).

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of synthesizing a compound of the formula (II) 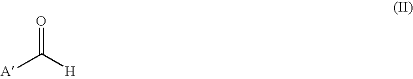
or a pharmaceutically acceptable salt thereof,
wherein A′ is 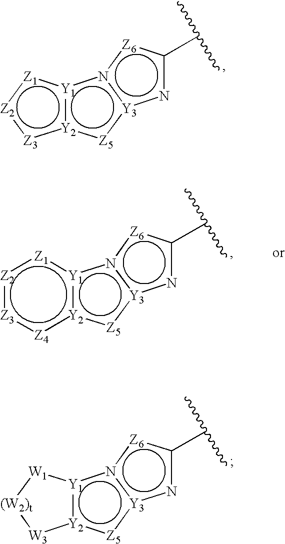
wherein
Z₁-Z₆ are each independently CR₂, N, O, S, or NR₁;
Y₁—Y₃ are each independently C, or N;
W₁—W₃ are each independently CR₄R₄, N, S, S(O), S(O)₂, or NR₁;
t is 1, 2, 3, or 4;
R₁ is hydrogen, —(C₁-C₆)-alkyl, -aryl, -heteroaryl, -heterocyclyl, —(C₃-C₇)-cycloalkyl, —(C₂-C₆)-alkenyl, —(C₂-C₆)-alkynyl, —(C₁-C₆)-perfluoroalkyl, —SO₂—(C₁-C₆)-alkyl, —SO₂—(C₁-C₆)-aryl, —C(O)-heteroaryl, —C(O)-aryl, —C(O)—(C₁-C₆)-alkyl, —C(O)—(C₃-C₇)-cycloalkyl, —C(O)-heterocyclyl, -aryl substituted with (C₁-C₆)-alkyl, -heteroaryl substituted with (C₁-C₆)-alkyl, —(C₁-C₆)-alkyl substituted with aryl, —(C₁-C₆)-alkyl substituted with heteroaryl, -heterocyclyl substituted with (C₁-C₆)-alkyl, —(C₂-C₈)-alkenyl substituted with aryl, —C(O)NR₆R₇, —SO₂NR₆R₇, —(C₁-C₆)-alkyl-O—(C₁-C₆)-alkyl-aryl, -aryl substituted with (C₁-C₆)-alkyl-O—(C₁-C₆)-alkyl, -heteroaryl substituted with (C₁-C₆)-alkyl-O—(C₁-C₆)-alkyl, —(C₁-C₆)-alkyl substituted with —O-aryl, —(C₁-C₆)-alkyl substituted with —O-heteroaryl, -aryl substituted with —O-aryl, -heteroaryl substituted with —O-heteroaryl, —(C₁-C₆)-alkyl-aryl-O-aryl, —(C₁-C₆)-alkyl-aryl-O-heteroaryl, —(C₁-C₆)-alkyl-aryl-O—(C₁-C₆)-alkyl-NR₆R₇, —C(O)O—(C₁-C₆)-alkyl, —C(O)O—(C₁-C₆)-aryl, or —C(O)O—(C₁-C₆)-heteroaryl; wherein each -alkyl, -alkenyl, -alkynyl, -cycloalkyl, -aryl, -heterocyclyl, or -heteroaryl is independently optionally substituted with one or more R₁₀;
R₂ is hydrogen, —(C₁-C₆)-alkyl, —(C₂-C₆)-alkenyl, —(C₂-C₆)-alkynyl, -halo, —CN, —NR₆R₇, —O—(C₁-C₆)-alkyl, —OH, -aryl, -heteroaryl, —CO₂R₆, —(C₁-C₆)-alkyl-aryl-O—(C₁-C₆)-alkyl-NR₆R₇, —O-aryl, —O-heteroaryl, —O—(C₃-C₆)-alkynyl, —(C₁-C₆)—O—(C₁-C₆)-alkyl-NR₆R₇, —O—(CH₂)₂—O—, -aryl-O—(C₁-C₆)-alkyl-NR₆R₇, —(C₁-C₆)-perfluoroalkyl, —S—(C₁-C₆)-alkyl, —S(O)—(C₁-C₆)-alkyl, —S(O)₂—(C₁-C₆)-alkyl, —S-aryl, —S(O)-aryl, —S(O)₂-aryl, —C(O)NR₆R₇, guanidino, cyclic guanidino, -aryl substituted with (C₁-C₆)-alkyl, —(C₁-C₆)-alkyl substituted with aryl, -heteroaryl substituted with (C₁-C₆)-alkyl, —(C₁-C₆)-alkyl substituted with heteroaryl, -heterocyclyl substituted with (C₁-C₆)-alkyl, —(C₂-C₈)-alkenyl substituted with aryl, —SO₂NR₆R₇, —(C₁-C₆)-alkyl-O—(C₁-C₆)-aryl, —(C₁-C₆)-alkyl-O-aryl, —(C₁-C₆)-alkyl-O-heteroaryl, -aryl substituted with —O-aryl, -heteroaryl substituted with —O-aryl,-aryl substituted with —O-heteroaryl, -heteroaryl substituted with —O-heteroaryl, —(C₁-C₆)-alkyl-aryl-O-aryl, —(C₁-C₆)-alkyl substituted with —O-aryl, —(C₁-C₆)-alkyl substituted with —O-heteroaryl, or —(C₁-C₆)-alkyl-aryl-O—(C₁-C₆)-alkyl-NR₆R₇; wherein each -alkyl, -alkenyl, -alkynyl, -cycloalkyl, -aryl, -heterocyclyl, or -heteroaryl is independently optionally substituted with one or more R₁₀;
R₁₀ is —NO₂, -aryl, -heteroaryl, —C(O)O—(C₁-C₆)-alkyl, —O—(C₁-C₆)-alkyl, —(C₁-C₆)-alkyl substituted with —O—(C₁-C₆)-alkyl, —O—(C₂-C₄)-alkyl substituted with —O—(C₁-C₆)-alkyl, —CN, -halo, —OH, —NR₁₁R₁₂, —CF₃, —OCF₃, —(C₁-C₆)-alkyl substituted with aryl, aryl substituted with (C₁-C₆)-alkyl, —(C₁-C₆)-alkyl substituted with —NR₁₁R₁₂, —(C₁-C₆)-alkyl substituted with —OH, —(C₁-C₆)-alkyl substituted with —O—(C₁-C₆)-alkyl, —S—(C₁-C₆)-alkyl, —SO₂NR₁₁R₁₂, —SO₂NHR₁₁, —CO₂H, —C(O)NR₁₀R₁₁, —O-aryl, —O-heteroaryl, —S-aryl, —S(O)-aryl, S(O)₂-aryl, —(C₁-C₆)-alkyl-O—(C₁C₆)-alkyl-NR₁₁R₁₂, —(C₁-C₆)-alkyl-aryl-O—(C₁-C₆)-alkyl-NR₁₁R₁₂, —(C₁-C₆)-alkyl, —(C₂-C₆)-alkenyl, —(C₂-C₆)-alkynyl, —(C₃-C₇)-cycloalkyl, —O—(C₁-C₆)-alkyl-O—(C₁-C₆)-alkyl, (C₁-C₆)-alkyl substituted with —NR₁₁R₁₂, —S-heteroaryl, —S(O)-heteroaryl, —S(O)₂-heteroaryl;
R₁₁ and R₁₂ are each independently hydrogen, —(C₁-C₆)-alkyl, -aryl, -heteroaryl, -aryl substituted with (C₁-C₆)-alkyl, —(C₁-C₆)-alkyl substituted with aryl, —(C₁-C₆)-alkyl substituted with heteroaryl, -heteroaryl substituted with (C₁-C₆)-alkyl; or R₁₁, R₁₂ and the nitrogen atom to which they are attached are taken together to form a three- to seven-membered saturated heterocyclic ring optionally having one or two additional heteroatoms selected from NR₁, O, S, S(O), or S(O)₂;
R₄ is hydrogen, —(C₁-C₆)-alkyl, —OH, —O—(C₁-C₆)-alkyl, —S—(C₁-C₆)-alkyl, —CO₂R₆, —NR₆R₇, —C(O)NR₆R₇; wherein each alkyl is independently optionally substituted with R₁₀; provided that two R₄ bound to the same carbon atom are not simultaneously —OH; or two R₄ bound to the same carbon atom are taken together to form ═O; or two R₄ and the carbon atom to which they are attached are taken together to form a five- to eight-membered spiro system optionally having up to three heteroatoms selected from NR₁, O, S, S(O), or S(O)₂;
R₆ and R₇ are each independently hydrogen, —(C₁-C₆)-alkyl, -aryl, -heteroaryl, -aryl substituted with (C₁-C₆)-alkyl, —(C₁-C₆)-alkyl substituted with aryl, —(C₁-C₆)-alkyl substituted with heteroaryl, -heteroaryl substituted with (C₁-C₆)-alkyl, wherein each -alkyl, -aryl, or -heteroaryl is optionally substituted with one or more R₁₀; or R₆, R₇ and the nitrogen to which they are attached are taken together to form a three- to seven-membered saturated heterocyclic ring optionally having one or two additional heteroatoms selected from NR₁, O, S, S(O), or S(O)₂;
comprising
a) reacting a compound of the formula (III)
D-NH₂ (III)
or a pharmaceutically acceptable salt thereof,
wherein D is 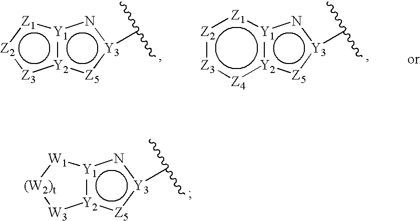
under conditions effective to bring about cyclization, thereby providing an aldehyde of the formula (II).

2. The method of claim 1, wherein step a) comprises reacting the compound of the formula (III) with a compound of the formula (VII) 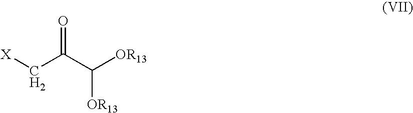
wherein
X is a leaving group and
R₁₃ is optionally substituted alkyl or optionally substituted aralkyl;
thereby providing an aldehyde of the formula (II).

3. The method of claim 2, wherein X is bromine, and R₁₃ is methyl.

4. The method of claim 1, wherein the compound of the formula (III) is present as its free base.

5. The method of claim 1, wherein the compound of the formula (III) is present as a pharmaceutically acceptable salt.

6. The method of claim 5, wherein the pharmaceutically acceptable salt of the compound of the formula (III) is a hydrochloride, salt, a hydroiodide salt, or a hydrobromide salt.

7. The method of claim 1, wherein step a) comprises reacting the compound of the formula (III) in the presence of a base.

8. The method of claim 7, wherein the base is sodium bicarbonate, potassium bicarbonate, pyridine, lutidine, triethylamine, diisopropylamine, or a combination thereof.

9. The method of claim 1, wherein step a) comprises reacting the compound of the formula (III) in the presence of a solvent selected from the group consisting of ethanol, 2-propanol, tetrahydrofuran, methylene chloride, acetonitrile, dimethylformamide, ethyl acetate, dichloroethane, dimethylacetamide, N-methylpyrrolidinone, acetone, or a combination thereof.

10. The method of claim 1, wherein the compound of formula (II) is

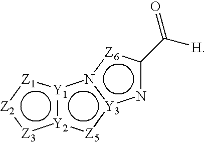

11. The method of claim 1, wherein the compound of formula (II) is

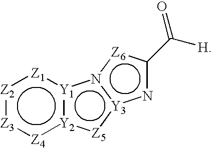

12. The method of claim 1, wherein the compound of formula (II) is

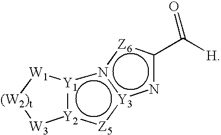

13. A method of synthesizing a compound of the formula (II) 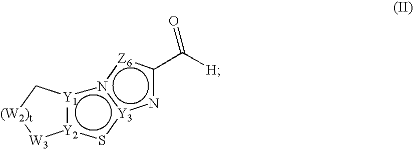
or a pharmaceutically acceptable salt thereof,
wherein
Z₆ is CR₂, N, O, S, or NR₁;
Y₁—Y₃ are each independently C, N;
W₂—W₃ are each independently CR₄R₄, S, S(O), S(O)₂, O, NR₁;
t is 1, 2, 3, or 4;
R₁ is hydrogen, —(C₁-C₆)-alkyl, -aryl, -heteroaryl, -heterocyclyl, —(C₃-C₇)-cycloalkyl, —(C₂-C₆)-alkenyl, —(C₂-C₆)-alkynyl, —(C₁-C₆)-perfluoroalkyl, —SO₂—(C₁-C₆)-alkyl, —SO₂—(C₁-C₆)-aryl, —C(O)-heteroaryl, —C(O)-aryl, —C(O)—(C₁-C₆)-alkyl, —C(O)—(C₃-C₇)-cycloalkyl, —C(O)-heterocyclyl, -aryl substituted with (C₁-C₆)-alkyl, -heteroaryl substituted with (C₁-C₆)-alkyl, —(C₁-C₆)-alkyl substituted with aryl, —(C₁₃-C₆)-alkyl substituted with heteroaryl, -heterocyclyl substituted with (C₁-C₆)-alkyl, —(C₂-C₈)-alkenyl substituted with aryl, —C(O)NR₆R₇, —SO₂NR₆R₇, —(C₁-C₆)-alkyl-O—(C₁-C₆)-alkyl-aryl, -aryl substituted with (C₁-C₆)-alkyl-O—(C₁-C₆)-alkyl, -heteroaryl substituted with (C₁-C₆)-alkyl-O—(C₁-C₆)-alkyl, —(C₁-C₆)-alkyl substituted with —O-aryl, —(C₁-C₆)-alkyl substituted with —O-heteroaryl, -aryl substituted with —O-aryl, -heteroaryl substituted with —O-heteroaryl, —(C₁-C₆)-alkyl-aryl-O-aryl, —(C₁-C₆)-alkyl-aryl-O-heteroaryl, —(C₁-C₆)-alkyl-aryl-O—(C₁-C₆)-alkyl-NR₆R₇, —C(O)O—(C₁-C₆)-alkyl, —C(O)O—(C₁-C₆)-aryl, or —C(O)O—(C₁-C₆)-heteroaryl;
R₂ is hydrogen, —(C₁-C₆)-alkyl, —(C₂-C₆)-alkenyl, —(C₂-C₆)-alkynyl, -halo, —CN, —NR₆R₇, —O—(C₁-C₆)-alkyl, —OH, -aryl, -heteroaryl, —CO₂R₆, —(C₁-C₆)-alkyl-aryl-O—(C₁-C₆)-alkyl-NR₆R₇, —O-aryl, —O-heteroaryl, —O—(C₃-C₆)-alkynyl, —(C₁-C₆)—O—(C₁-C₆)-alkyl-NR₆R₇, —O—(CH₂)₂—O—, -aryl-O—(C₁-C₆)-alkyl-NR₆R₇, —(C₁-C₆)-perfluoroalkyl, —S—(C₁-C₆)-alkyl, —S(O)—(C₁-C₆)-alkyl, —S(O)₂—(C₁-C₆)-alkyl, —S-aryl, —S(O)-aryl, —S(O)₂-aryl, —C(O)NR₆R₇, guanidino, cyclic guanidino, -aryl substituted with (C₁-C₆)-alkyl, —(C₁-C₆)-alkyl substituted with aryl, -heteroaryl substituted with (C₁-C₆)-alkyl, —(C₁-C₆)-alkyl substituted with heteroaryl, -heterocyclyl substituted with (C₁-C₆)-alkyl, —(C₂-C₈)-alkenyl substituted with aryl, —SO₂NR₆R₇, —(C₁-C₆)-alkyl-O—(C₁-C₆)-aryl, —(C₁-C₆)-alkyl-O-aryl, —(C₁-C₆)-alkyl-O-heteroaryl, -aryl substituted with —O-aryl, -heteroaryl substituted with —O-aryl,-aryl substituted with —O-heteroaryl, -heteroaryl substituted with —O-heteroaryl, —(C₁-C₆)-alkyl-aryl-O-aryl, —(C₁-C₆)-alkyl substituted with —O-aryl, —(C₁-C₆)-alkyl substituted with —O-heteroaryl, or —(C₁-C₆)-alkyl-aryl-O—(C₁-C₆)-alkyl-NR₆R₇; wherein each -alkyl, -alkenyl, -alkynyl, -cycloalkyl, -aryl, -heterocyclyl, or -heteroaryl is independently optionally substituted with one or more R₁₀;
R₁₀ is —NO₂, -aryl, -heteroaryl, —C(O)O—(C₁-C₆)-alkyl, —O—(C₁-C₆)-alkyl, —(C₁-C₆)-alkyl substituted with —O—(C₁-C₆)-alkyl, —O—(C₂-C₄)-alkyl substituted with —O—(C₁-C₆)-alkyl, —CN, -halo, —OH, —NR₁₁R₁₂, —CF₃, —OCF₃, —(C₁-C₆)-alkyl substituted with aryl, aryl substituted with (C₁-C₆)-alkyl, —(C₁-C₆)-alkyl substituted with —NR₁₁R₁₂, —(C₁-C₆)-alkyl substituted with —OH, —(C₁-C₆)-alkyl substituted with —O—(C₁-C₆)-alkyl, —S—(C₁-C₆)-alkyl, —SO₂NR₁₁R₁₂, —SO₂NHR₁₁, —CO₂H, —C(O)NR₁₀R₁₁, —O-aryl, —O-heteroaryl, —S-aryl, —S(O)-aryl, S(O)₂-aryl, —(C₁-C₆)-alkyl-O—(C₁C₆)-alkyl-NR₁₁R₁₂, —(C₁-C₆)-alkyl-aryl-O—(C₁-C₆)-alkyl-NR₁₁R₁₂, —(C₁-C₆)-alkyl, —(C₂-C₆)-alkenyl, —(C₂-C₆)-alkynyl, —(C₃-C₇)-cycloalkyl, —O—(C₁-C₆)-alkyl-O—(C₁-C₆)-alkyl, (C₁-C₆)-alkyl substituted with —NR₁₁R₁₂, —S-heteroaryl, —S(O)-heteroaryl, —S(O)₂-heteroaryl;
R₁₁ and R₁₂ are each independently hydrogen, —(C₁-C₆)-alkyl, -aryl, -heteroaryl, -aryl substituted with (C₁-C₆)-alkyl, —(C₁-C₆)-alkyl substituted with aryl, —(C₁-C₆)-alkyl substituted with heteroaryl, -heteroaryl substituted with (C₁-C₆)-alkyl; or R₁₁, R₁₂ and the nitrogen atom to which they are attached are taken together to form a three- to seven-membered saturated heterocyclic ring optionally having one or two additional heteroatoms selected from NR₁, O, S, S(O), or S(O)₂;
R₄ is hydrogen, —(C₁-C₆)-alkyl, —OH, —O—(C₁-C₆)-alkyl, —S—(C₁-C₆)-alkyl, —CO₂R₆, —NR₆R₇, —C(O)NR₆R₇; wherein each alkyl is independently optionally substituted with R₁₀; provided that two R₄ bound to the same carbon atom are not simultaneously —OH; or two R₄ bound to the same carbon atom are taken together to form ═O; or two R₄ and the carbon atom to which they are attached are taken together to form a five- to eight-membered spiro system optionally having up to three heteroatoms selected from NR₁, O, S, S(O), or S(O)₂;
R₆ and R₇ are each independently hydrogen, —(C₁-C₆)-alkyl, -aryl, -heteroaryl, -aryl substituted with (C₁-C₆)-alkyl, —(C₁-C₆)-alkyl substituted with aryl, —(C₁-C₆)-alkyl substituted with heteroaryl, -heteroaryl substituted with (C₁-C₆)-alkyl, wherein each -alkyl, -aryl, or -heteroaryl is optionally substituted with one or more R₁₀; or R₆, R₇ and the nitrogen to which they are attached are taken together to form a three- to seven-membered saturated heterocyclic ring optionally having one or two additional heteroatoms selected from NR₁, O, S, S(O), or S(O)₂;
comprising
a) reacting a compound of the formula (VII) 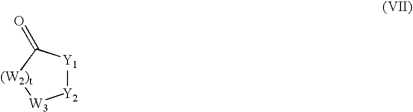
or a pharmaceutically acceptable salt thereof,
under conditions effective to bring about cyclization, thereby providing an amine of the formula (III) 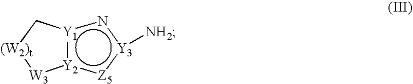
or a pharmaceutically acceptable salt thereof,
b) reacting a compound of the formula (III) under conditions effective to bring about cyclization, thereby providing an aldehyde of the formula (II).

14. The method of claim 13, wherein step b) comprises reacting the compound of the formula (III) with a compound of the formula (VII) 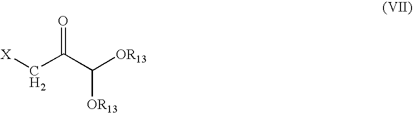
wherein
X is a leaving group and
R₁₃ is optionally substituted alkyl or optionally substituted arylkyl;
thereby providing an aldehyde of the formula (II).

15. The method of claim 14, wherein X is Br and R₁₃ is methyl.

16. The method of claim 13, wherein the compound of the formula (III) is present as its free base.

17. The method of claim 13, wherein the compound of the formula (III) is present as a pharmaceutically acceptable salt.

18. The method of claim 17, wherein the pharmaceutically acceptable salt of the compound of the formula (III) is a hydrochloride salt, a hydroiodide salt, or a hydrobromide salt.

19. The method of claim 13, wherein step b) comprises reacting the compound of the formula (III) in the presence of a base.

20. The method of claim 19, wherein the base is sodium bicarbonate, potassium bicarbonate, pyridine, lutidine, triethylamine, diisopropylethylamine, or a combination thereof.

21. The method of claim 13, wherein step b) comprises reacting the compound of the formula (III) in the presence of a solvent selected from the group consisting of ethanol, 2-propanol, tetrahydrofuran, methylene chloride, acetonitrile, dimethylformamide, ethyl acetate, dichloroethane, dimethylacetamide, N-methylpyrrolidinone, acetone, or a combination thereof.

22. The method of claim 13, wherein the compound of formula (II) is

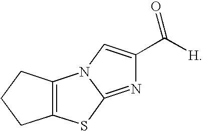

23. A method of synthesizing a compound of the formula (VII) 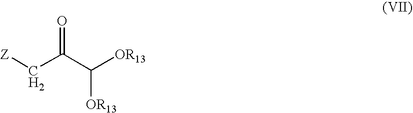
wherein Z is halogen and R₁₃ is an optionally substituted alkyl or aralkyl group, comprising reacting a compound of the formula (VIII) 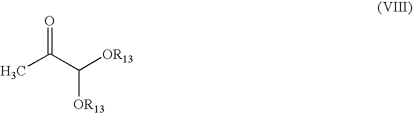
in the presence of a halogenating agent and from about 1% to about 100% (v/v) methanol in acetonitrile, thereby providing a compound of the formula (VII).

24. The method of claim 23, wherein Z is Br and R₁₃ is methyl, and a compound of formula (VIII) is reacted in the presence of a brominating agent, thereby providing a compound of the formula (VII).

25. The method of claim 23, comprising from about 1% to about 20% methanol in acetonitrile.

26. The method of claim 23, comprising from about 2% to about 10% methanol in acetonitrile.

27. The method of claim 23, comprising from about 3% to about 8% methanol in acetonitrile.

28. The method of claim 23, comprising about 5% methanol in acetonitrile.

29. The method of claim 24, wherein the brominating agent is Br₂ or N-bromosuccinimide.

30. A method of synthesizing a compound of the formula (I) 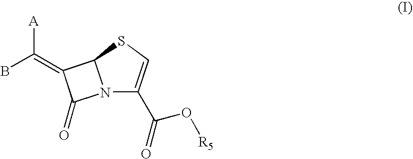
or a pharmaceutically acceptable salt thereof,
wherein
one of A and B is hydrogen and the other of A and B is 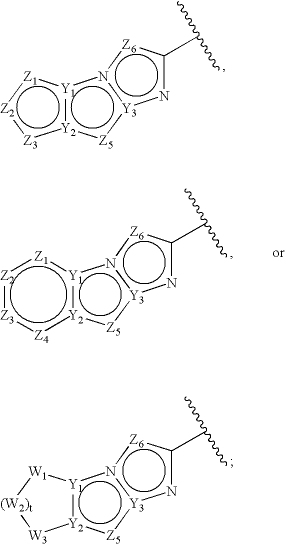
R₅ is hydrogen, —(C₁-C₆)-alkyl, —(C₅-C₆)-cycloalkyl, —CH(R₃)—O—C(O)—(C₁-C₆)-alkyl, benzyhydryl, or p-nitrobenzoyl;
Z₁-Z₆ are each independently CR₂, N, O, S, or NR₁;
Y₁—Y₃ are each independently C, or N;
W₁—W₃ are each independently CR₄R₄ S, S(O), S(O)₂, O, or NR₁;
t is 1, 2, 3, or 4;
R₁ is hydrogen, —(C₁-C₆)-alkyl, -aryl, -heteroaryl, -heterocyclyl, —(C₃-C₇)-cycloalkyl, —(C₂-C₆)-alkenyl, —(C₂-C₆)-alkynyl, —(C₁-C₆)-perfluoroalkyl, —SO₂—(C₁-C₆)-alkyl, —SO₂—(C₁-C₆)-aryl, —C(O)-heteroaryl, —C(O)-aryl, —C(O)—(C₁-C₆)-alkyl, —C(O)—(C₃-C₇)-cycloalkyl, —C(O)-heterocyclyl, -aryl substituted with (C₁-C₆)-alkyl, -heteroaryl substituted with (C₁-C₆)-alkyl, —(C₁-C₆)-alkyl substituted with aryl, —(C₁₃-C₆)-alkyl substituted with heteroaryl, -heterocyclyl substituted with (C₁-C₆)-alkyl, —(C₂-C₈)-alkenyl substituted with aryl, —C(O)NR₆R₇, —SO₂NR₆R₇, —(C₁-C₆)-alkyl-O—(C₁-C₆)-alkyl-aryl, -aryl substituted with (C₁-C₆)-alkyl-O—(C₁-C₆)-alkyl, -heteroaryl substituted with (C₁-C₆)-alkyl-O—(C₁-C₆)-alkyl, —(C₁-C₆)-alkyl substituted with —O-aryl, —(C₁-C₆)-alkyl substituted with —O-heteroaryl, -aryl substituted with —O-aryl, -heteroaryl substituted with —O-heteroaryl, —(C₁-C₆)-alkyl-aryl-O-aryl, —(C₁-C₆)-alkyl-aryl-O-heteroaryl, —(C₁-C₆)-alkyl-aryl-O—(C₁-C₆)-alkyl-NR₆R₇, —C(O)O—(C₁-C₆)-alkyl, —C(O)O—(C₁-C₆)-aryl, or —C(O)O—(C₁-C₆)-heteroaryl; wherein each -alkyl, -alkenyl, -alkynyl, -cycloalkyl, -aryl, -heterocyclyl, or -heteroaryl is independently optionally substituted with one or more R₁₀;
R₂ is hydrogen, —(C₁-C₆)-alkyl, —(C₂-C₆)-alkenyl, —(C₂-C₆)-alkynyl, -halo, —CN, —NR₆R₇, —O—(C₁-C₆)-alkyl, —OH, -aryl, -heteroaryl, —CO₂R₆, —(C₁-C₆)-alkyl-aryl-O—(C₁-C₆)-alkyl-NR₆R₇, —O-aryl, —O-heteroaryl, —O—(C₃-C₆)-alkynyl, —(C₁-C₆)—O—(C₁-C₆)-alkyl-NR₆R₇, —O—(CH₂)₂—O—, -aryl-O—(C₁-C₆)-alkyl-NR₆R₇, —(C₁-C₆)-perfluoroalkyl, —S—(C₁-C₆)-alkyl, —S(O)—(C₁-C₆)-alkyl, —S(O)₂—(C₁-C₆)-alkyl, —S-aryl, —S(O)-aryl, —S(O)₂-aryl, —C(O)NR₆R₇, guanidino, cyclic guanidino, -aryl substituted with (C₁-C₆)-alkyl, —(C₁-C₆)-alkyl substituted with aryl, -heteroaryl substituted with (C₁-C₆)-alkyl, —(C₁-C₆)-alkyl substituted with heteroaryl, -heterocyclyl substituted with (C₁-C₆)-alkyl, —(C₂-C₈)-alkenyl substituted with aryl, —SO₂NR₆R₇, —(C₁-C₆)-alkyl-O—(C₁-C₆)-aryl, —(C₁-C₆)-alkyl-O-aryl, —(C₁-C₆)-alkyl-O-heteroaryl, -aryl substituted with —O-aryl, -heteroaryl substituted with —O-aryl,-aryl substituted with —O-heteroaryl, -heteroaryl substituted with —O-heteroaryl, —(C₁-C₆)-alkyl-aryl-O-aryl, —(C₁-C₆)-alkyl substituted with —O-aryl, —(C₁-C₆)-alkyl substituted with —O-heteroaryl, or —(C₁-C₆)-alkyl-aryl-O—(C₁-C₆)-alkyl-NR₆R₇; wherein each -alkyl, -alkenyl, -alkynyl, -cycloalkyl, -aryl, -heterocyclyl, or -heteroaryl is independently optionally substituted with one or more R₁₀;
R₁₀ is —NO₂, -aryl, -heteroaryl, —C(O)O—(C₁-C₆)-alkyl, —O—(C₁-C₆)-alkyl, —(C₁-C₆)-alkyl substituted with —O—(C₁-C₆)-alkyl, —O—(C₂-C₄)-alkyl substituted with —O—(C₁-C₆)-alkyl, —CN, -halo, —OH, —NR₁₁R₁₂, —CF₃, —OCF₃, —(C₁-C₆)-alkyl substituted with aryl, aryl substituted with (C₁-C₆)-alkyl, —(C₁-C₆)-alkyl substituted with —NR₁₁R₁₂, —(C₁-C₆)-alkyl substituted with —OH, —(C₁-C₆)-alkyl substituted with —O—(C₁-C₆)-alkyl, —S—(C₁-C₆)-alkyl, —SO₂NR₁₁R₁₂, —SO₂NHR₁₁, —CO₂H, —C(O)NR₁₀R₁₁, —O-aryl, —O-heteroaryl, —S-aryl, —S(O)-aryl, S(O)₂-aryl, —(C₁-C₆)-alkyl-O—(C₁C₆)-alkyl-NR₁₁R₁₂, —(C₁-C₆)-alkyl-aryl-O—(C₁-C₆)-alkyl-NR₁₁R₁₂, —(C₁-C₆)-alkyl, —(C₂-C₆)-alkenyl, —(C₂-C₆)-alkynyl, —(C₃-C₇)-cycloalkyl, —O—(C₁-C₆)-alkyl-O—(C₁-C₆)-alkyl, (C₁-C₆)-alkyl substituted with —NR₁₁R₁₂, —S-heteroaryl, —S(O)-heteroaryl, —S(O)₂-heteroaryl;
R₁₁ and R₁₂ are each independently hydrogen, —(C₁-C₆)-alkyl, -aryl, -heteroaryl, -aryl substituted with (C₁-C₆)-alkyl, —(C₁-C₆)-alkyl substituted with aryl, —(C₁-C₆)-alkyl substituted with heteroaryl, -heteroaryl substituted with (C₁-C₆)-alkyl; or R₁₁, R₁₂ and the nitrogen atom to which they are attached are taken together to form a three- to seven-membered saturated heterocyclic ring optionally having one or two additional heteroatoms selected from NR₁, O, S, S(O), or S(O)₂;
R₃ is hydrogen, —(C₁-C₆)-alkyl, —(C₅-C₆)-cycloalkyl, -aryl, or -heteroaryl; wherein each -aryl or -heteroaryl is independently optionally substituted with R₁₀;
R₄ is hydrogen, —(C₁-C₆)-alkyl, —OH, —O—(C₁-C₆)-alkyl, —S—(C₁-C₆)-alkyl, —CO₂R₆, —NR₆R₇, —C(O)NR₆R₇; wherein each alkyl is independently optionally substituted with R₁₀; provided that two R₄ bound to the same carbon atom are not simultaneously —OH; or two R₄ bound to the same carbon atom are taken together to form ═O; or two R₄ and the carbon atom to which they are attached are taken together to form a five- to eight-membered spiro system optionally having up to three heteroatoms selected from NR₁, O, S, S(O), or S(O)₂;
R₆ and R₇ are each independently hydrogen, —(C₁-C₆)-alkyl, -aryl, -heteroaryl, -aryl substituted with (C₁-C₆)-alkyl, —(C₁-C₆)-alkyl substituted with aryl, —(C₁-C₆)-alkyl substituted with heteroaryl, -heteroaryl substituted with (C₁-C₆)-alkyl, wherein each -alkyl, -aryl, or -heteroaryl is optionally substituted with one or more R₁₀; or R₆, R₇ and the nitrogen to which they are attached are taken together to form a three- to seven-membered saturated heterocyclic ring optionally having one or two additional heteroatoms selected from NR₁, O, S, S(O), or S(O)₂;
comprising
a) reacting a compound of the formula (III)
D-NH₂ (III)
or a pharmaceutically acceptable salt thereof,
wherein D is 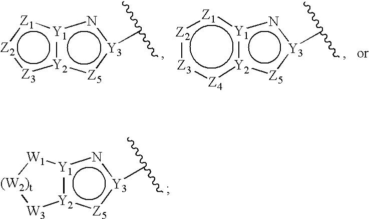
under conditions effective to bring about cyclization, thereby providing an aldehyde of the formula (II): 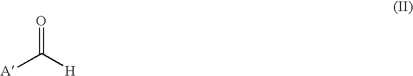
or a pharmaceutically acceptable salt thereof,
wherein A′ is A or B, whichever of A or B is not hydrogen;
b) condensing the aldehyde of formula (II) with a 6-bromo-penem of the formula (IV) 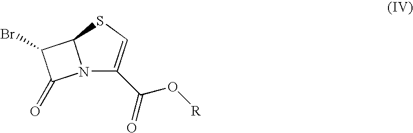
or a pharmaceutically acceptable salt thereof,
where R is a protecting group;
under conditions effective to provide an aldol of the formula (V): 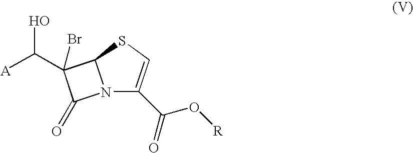
or a pharmaceutically acceptable salt thereof,
c) reacting the aldol of formula (V) under conditions effective to provide a compound of the formula (VI): 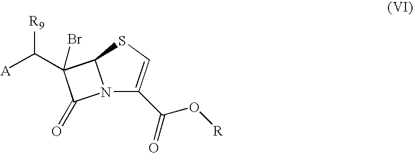
wherein R₉ is —X₁ or —OR₈;
X₁ is —Br, —I, or —Cl; and
R₈ is —SO₂-alkyl, —SO₂-aryl, —C(O)-alkyl, or —C(O)-aryl;
d) subjecting the compound of formula (VI) to conditions effective to bring about reductive elimination, thereby forming a compound of the formula (I).

31. The method of claim 30, wherein step a) comprises reacting the compound of the formula (III) with a compound of the formula (VII) 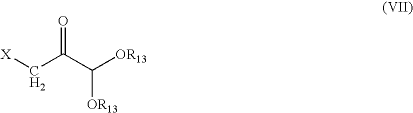
wherein
X is a leaving group and
R₁₃ is optionally substituted alkyl or optionally substituted arylkyl;
thereby providing an aldehyde of the formula (II).

32. The method of claim 31, wherein X is Br and R₁₃ is methyl.

33. The method of claim 30, wherein the compound of the formula (III) is present as its free base.

34. The method of claim 30, wherein the compound of the formula (III) is present as a pharmaceutically acceptable salt.

35. The method of claim 34, wherein the pharmaceutically acceptable salt of the compound of formula (III) is a hydrochloride salt, a hydroiodide salt, or a hydrobromide salt.

36. The method of claim 30, wherein step a) comprises reacting the compound of the formula (III) in the presence of a base.

37. The method of claim 36, wherein the base is sodium bicarbonate, potassium bicarbonate, pyridine, lutidine, triethylamine, diisopropylethylamine, or a combination thereof.

38. The method of claim 30, wherein step a) comprises reacting the compound of the formula (III) in the presence of a solvent selected from the group consisting of ethanol, 2-propanol, tetrahydrofuran, methylene chloride, acetonitrile, dimethylformamide, ethyl acetate, dichloroethane, dimethylacetamide, N-methylpyrrolidinone, acetone, or a combination thereof.

39. The method of claim 30, wherein the compound of formula (II) is

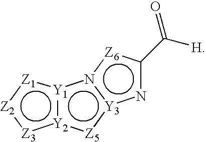

40. The method of claim 30, wherein the compound of formula (II) is

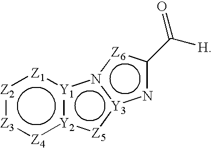

41. The method of claim 30, wherein the compound of formula (II) is

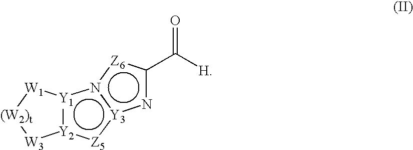

42. The method of claim 30, wherein the protecting group is p-nitrobenzyl, benzyl, para-methoxy benzyl, benzylhydrol, or trityl.

43. A compound of the formula (I): 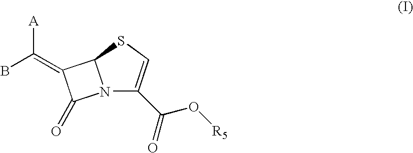
or a pharmaceutically acceptable salt thereof,
wherein
one of A and B is hydrogen and the other of A and B is 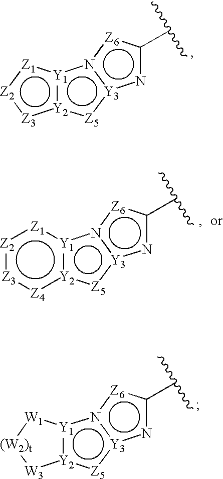
wherein
R₅ is hydrogen, —(C₁-C₆)-alkyl, —(C₅-C₆)-cycloalkyl, or —CH(R₃)—O—C(O)—(C₁ -C₆)-alkyl;
Z₁-Z₆ are each independently CR₂, N, O, S, or NR₁;
Y₁—Y₃ are each independently C, or N;
W₁—W₃ are each independently CR₄R₄, S, S(O), S(O)₂, O, or NR₁;
t is 1, 2, 3, or 4;
R₁ is hydrogen, —(C₁-C₆)-alkyl, -aryl, -heteroaryl, -heterocyclyl, —(C₃-C₇)-cycloalkyl, —(C₂-C₆)-alkenyl, —(C₂-C₆)-alkynyl, —(C₁-C₆)-perfluoroalkyl, —SO₂—(C₁-C₆)-alkyl, —SO₂—(C₁-C₆)-aryl, —C(O)-heteroaryl, —C(O)-aryl, —C(O)—(C₁-C₆)-alkyl, —C(O)—(C₃-C₇)-cycloalkyl, —C(O)-heterocyclyl, -aryl substituted with (C₁-C₆)-alkyl, -heteroaryl substituted with (C₁-C₆)-alkyl, —(C₁-C₆)-alkyl substituted with aryl, —(C₁₃-C₆)-alkyl substituted with heteroaryl, -heterocyclyl substituted with (C₁-C₆)-alkyl, —(C₂-C₈)-alkenyl substituted with aryl, —C(O)NR₆R₇, —SO₂NR₆R₇, —(C₁-C₆)-alkyl-O—(C₁-C₆)-alkyl-aryl, -aryl substituted with (C₁-C₆)-alkyl-O—(C₁-C₆)-alkyl, -heteroaryl substituted with (C₁-C₆)-alkyl-O—(C₁-C₆)-alkyl, —(C₁-C₆)-alkyl substituted with —O-aryl, —(C₁-C₆)-alkyl substituted with —O-heteroaryl, -aryl substituted with —O-aryl, -heteroaryl substituted with —O-heteroaryl, —(C₁-C₆)-alkyl-aryl-O-aryl, —(C₁-C₆)-alkyl-aryl-O-heteroaryl, —(C₁-C₆)-alkyl-aryl-O—(C₁-C₆)-alkyl-NR₆R₇, —C(O)O—(C₁-C₆)-alkyl, —C(O)O—(C₁-C₆)-aryl, or —C(O)O—(C₁-C₆)-heteroaryl; wherein each -alkyl, -alkenyl, -alkynyl, -cycloalkyl, -aryl, -heterocyclyl, or -heteroaryl is independently optionally substituted with one or more R₁₀;
R₂ is hydrogen, —(C₁-C₆)-alkyl, —(C₂-C₆)-alkenyl, —(C₂-C₆)-alkynyl, -halo, —CN, —NR₆R₇, —O—(C₁-C₆)-alkyl, —OH, -aryl, -heteroaryl, —CO₂R₆, —(C₁-C₆)-alkyl-aryl-O—(C₁-C₆)-alkyl-NR₆R₇, —O-aryl, —O-heteroaryl, —O—(C₃-C₆)-alkynyl, —(C₁-C₆)—O—(C₁-C₆)-alkyl-NR₆R₇, —O—(CH₂)₂—O—, -aryl-O—(C₁-C₆)-alkyl-NR₆R₇, —(C₁-C₆)-perfluoroalkyl, —S—(C₁-C₆)-alkyl, —S(O)—(C₁-C₆)-alkyl, —S(O)₂—(C₁-C₆)-alkyl, —S-aryl, —S(O)-aryl, —S(O)₂-aryl, —C(O)NR₆R₇, guanidino, cyclic guanidino, -aryl substituted with (C₁-C₆)-alkyl, —(C₁-C₆)-alkyl substituted with aryl, -heteroaryl substituted with (C₁-C₆)-alkyl, —(C₁-C₆)-alkyl substituted with heteroaryl, -heterocyclyl substituted with (C₁-C₆)-alkyl, —(C₂-C₈)-alkenyl substituted with aryl, —SO₂NR₆R₇, —(C₁-C₆)-alkyl-O—(C₁-C₆)-aryl, —(C₁-C₆)-alkyl-O-aryl, —(C₁-C₆)-alkyl-O-heteroaryl, -aryl substituted with —O-aryl, -heteroaryl substituted with —O-aryl,-aryl substituted with —O-heteroaryl, -heteroaryl substituted with —O-heteroaryl, —(C₁-C₆)-alkyl-aryl-O-aryl, —(C₁-C₆)-alkyl substituted with —O-aryl, —(C₁-C₆)-alkyl substituted with —O-heteroaryl, or —(C₁-C₆)-alkyl-aryl-O—(C₁-C₆)-alkyl-NR₆R₇; wherein each -alkyl, -alkenyl, -alkynyl, -cycloalkyl, -aryl, -heterocyclyl, or -heteroaryl is independently optionally substituted with one or more R₁₀;
R₁₀ is —NO₂, -aryl, -heteroaryl, —C(O)O—(C₁-C₆)-alkyl, —O—(C₁-C₆)-alkyl, —(C₁-C₆)-alkyl substituted with —O—(C₁-C₆)-alkyl, —O—(C₂-C₄)-alkyl substituted with —O—(C₁-C₆)-alkyl, —CN, -halo, —OH, —NR₁₁R₁₂, —CF₃, —OCF₃, —(C₁-C₆)-alkyl substituted with aryl, aryl substituted with (C₁-C₆)-alkyl, —(C₁-C₆)-alkyl substituted with —NR₁₁R₁₂, —(C₁-C₆)-alkyl substituted with —OH, —(C₁-C₆)-alkyl substituted with —O—(C₁-C₆)-alkyl, —S—(C₁-C₆)-alkyl, —SO₂NR₁₁R₁₂, —SO₂NHR₁₁, —CO₂H, —C(O)NR₁₀R₁₁, —O-aryl, —O-heteroaryl, —S-aryl, —S(O)-aryl, S(O)₂-aryl, —(C₁-C₆)-alkyl-O—(C₁C₆)-alkyl-NR₁₁R₁₂, —(C₁-C₆)-alkyl-aryl-O—(C₁-C₆)-alkyl-NR₁₁R₁₂, —(C₁-C₆)-alkyl, —(C₂-C₆)-alkenyl, —(C₂-C₆)-alkynyl, —(C₃-C₇)-cycloalkyl, —O—(C₁-C₆)-alkyl-O—(C₁-C₆)-alkyl, (C₁-C₆)-alkyl substituted with —NR₁₁R₁₂, —S-heteroaryl, —S(O)-heteroaryl, —S(O)₂-heteroaryl;
R₁₁ and R₁₂ are each independently hydrogen, —(C₁-C₆)-alkyl, -aryl, -heteroaryl, -aryl substituted with (C₁-C₆)-alkyl, —(C₁-C₆)-alkyl substituted with aryl, —(C₁-C₆)-alkyl substituted with heteroaryl, -heteroaryl substituted with (C₁-C₆)-alkyl; or R₁₁, R₁₂ and the nitrogen atom to which they are attached are taken together to form a three- to seven-membered saturated heterocyclic ring optionally having one or two additional heteroatoms selected from NR₁, O, S, S(O), or S(O)₂;
R₃ is hydrogen, —(C₁-C₆)-alkyl, —(C₅-C₆)-cycloalkyl, -aryl, or -heteroaryl; wherein each -aryl or -heteroaryl is independently optionally substituted with R₁₀;
R₄ is hydrogen, —(C₁-C₆)-alkyl, —OH, —O—(C₁-C₆)-alkyl, —S—(C₁-C₆)-alkyl, —CO₂R₆, —NR₆R₇, —C(O)NR₆R₇; wherein each alkyl is independently optionally substituted with R₁₀; provided that two R₄ bound to the same carbon atom are not simultaneously —OH; or two R₄ bound to the same carbon atom are taken together to form ═O; or two R₄ and the carbon atom to which they are attached are taken together to form a five- to eight-membered spiro system optionally having up to three heteroatoms selected from NR₁, O, S, S(O), or S(O)₂;
R₆ and R₇ are each independently hydrogen, —(C₁-C₆)-alkyl, -aryl, -heteroaryl, -aryl substituted with (C₁-C₆)-alkyl, —(C₁-C₆)-alkyl substituted with aryl, —(C₁-C₆)-alkyl substituted with heteroaryl, -heteroaryl substituted with (C₁-C₆)-alkyl, wherein each -alkyl, -aryl, or -heteroaryl is optionally substituted with one or more R₁₀; or R₆, R₇ and the nitrogen to which they are attached are taken together to form a three- to seven-membered saturated heterocyclic ring optionally having one or two additional heteroatoms selected from NR₁, O, S, S(O), or S(O)₂;
prepared by the method comprising:
a) reacting a compound of the formula (III)
D-NH₂ (III)
or a pharmaceutically acceptable salt thereof,
wherein D is 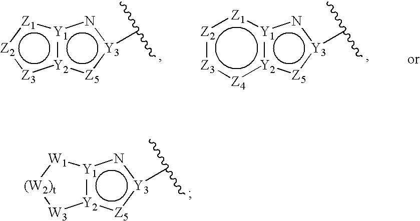
under conditions effective to bring about cyclization, thereby providing an aldehyde of the formula (II): 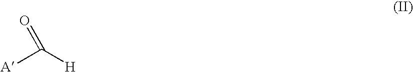
or a pharmaceutically acceptable salt thereof,
wherein A′ is A or B, whichever of A or B is not hydrogen;
b) condensing the aldehyde of formula (II) with a 6-bromo-penem of the formula (IV) 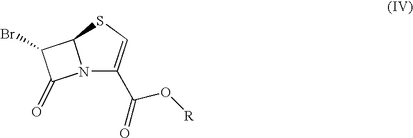
or a pharmaceutically acceptable salt thereof,
where R is a protecting group;
under conditions effective to provide an aldol of the formula (V): 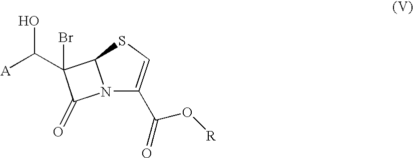
or a pharmaceutically acceptable salt thereof,
c) reacting the aldol of formula (V) under conditions effective to provide a compound of the formula (VI): 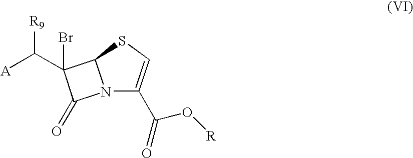
or a pharmaceutically acceptable salt thereof,
wherein R₉ is —X₁ or —OR₈;
X₁ is —Br, —I, or —Cl; and
R₈ is —SO₂-alkyl, —SO₂-aryl, —C(O)-alkyl, or —C(O)-aryl;
d) subjecting the compound of formula (VI) to conditions effective to bring about reductive elimination, thereby forming a compound of the formula (I).
